(19) United States
(12) Patent Application Publication
(10) Pub. No.: US 2006/0258706 A1
(43) Pub. Date: Nov. 16, 2006

(54) Solid forms of a JNK inhibitor

(76) Inventors: Manohar Saindane, Monmouth Junction, NJ (US); Chuansheng Ge, Belle Mead, NJ (US)

Correspondence Address: JONES DAY, 222 EAST 41ST ST, NEW YORK, NY, 10017, US

(21) Appl. No.: 11/414,630

(22) Filed: Apr. 27, 2006

Related U.S. Application Data
(60) Provisional application No. 60/676,693, filed on Apr. 29, 2005.

(51) Int. Cl. A61K 31/454; C07D 403/14

(52) U.S. Cl. 514/322; 546/199

(57) ABSTRACT

The present invention provides solid forms of Compound (I), pharmaceutical compositions thereof, and methods for the treatment or prevention of diseases including, but not limited to a liver disease, cancer, a cardiovascular disease, a metabolic disease, a renal disease, an autoimmune condition, an inflammatory condition, macular degeneration, pain and related syndromes, disease-related wasting, an asbestos-related condition, pulmonary hypertension, ischemia/reperfusion injury, central nervous system (CNS) injury/damage or a disease treatable or preventable by the inhibition of JNK. In particular, the invention relates to certain novel crystal forms of the compound 1-(5-(1H-1,2,4-triazol-5-yl)(1H-indazol-3-yl))-3-(2-piperidylethoxy)benzene.

This application claims the priority benefit of U.S. application No. 60/676,693, filed Apr. 29, 2005, the disclosure of which is incorporated by reference herein in its entirety.

1. FIELD OF THE INVENTION

The present invention relates to solid forms of a Jun N-terminal kinase (“JNK”) inhibitor, compositions comprising the solid forms, methods of making the solid forms, and methods for their use for the treatment or prevention of diseases including, but not limited to, a liver disease, cancer, a cardiovascular disease, a metabolic disease, a renal disease, an autoimmune condition, an inflammatory condition, macular degeneration, pain and related syndromes, disease-related wasting, an asbestos-related condition, pulmonary hypertension, ischemia/reperfusion injury, central nervous system (CNS) injury/damage or a disease treatable or preventable by the inhibition of JNK. In particular, the invention relates to certain novel crystal forms of the compound 1-(5-(1H-1,2,4-triazol-5-yl)(1H-indazol-3-yl))-3-(2-piperidylethoxy)benzene.

### 2. BACKGROUND OF THE INVENTION

Three JNK enzymes have been identified as products of distinct genes (Hibi et al, supra; Mohit et al., supra). Ten different isoforms of JNK have been identified. These represent alternatively spliced forms of three different genes: JNK1, JNK2 and JNK3. JNK1 and 2 are ubiquitously expressed in human tissues, whereas JNK3 is selectively expressed in the brain, heart and testis (Dong C., Yang D., Wysk M., Whitmarsh A., Davis R., Flavell R. Science 270:1-4, 1998). JNK binds to the N-terminal region of c-jun and ATF-2 and phosphorylates two sites within the activation domain of each transcription factor (Hibi M., Lin A., Smeal T, Minden A., Karin M. Genes Dev. 7:2135-2148, 1993; Mohit A. A., Martin M. H., and Miller C. A. Neuron 14:67-75, 1995).

The JNK pathway is activated by exposure of cells to environmental stress or by treatment of cells with pro-inflammatory cytokines. Targets of the JNK pathway include the transcription factors c-jun and ATF2 (Whitmarsh A. J., and Davis R. J. J. Mol. Med. 74:589-607, 1996). These transcription factors are members of the basic leucine zipper (bZIP) group that bind as homo- and hetero-dimeric complexes to AP-1 and AP-1-like sites in the promoters of many genes (Karin M., Liu Z. G. and Zandi E. Curr. Opin. Cell Biol. 9:240-246, 1997).

Activation of the JNK pathway has been documented in a number of disease settings, providing the rationale for targeting this pathway for drug discovery. In addition, molecular genetic approaches have validated the pathogenic role of this pathway in several diseases. For example, autoimmune and inflammatory diseases arise from the over-activation of the immune system. Activated immune cells express many genes encoding inflammatory molecules, including cytokines, growth factors, cell surface receptors, cell adhesion molecules and degradative enzymes. Many of these genes are regulated by the JNK pathway, through activation of the transcription factors AP-1 and ATF-2, including TNFα, IL-2, E-selectin and matrix metalloproteinases such as collagenase-1 (Manning A. M. and Mercurio F. Exp. Opin Invest. Drugs 6: 555-567, 1997).

In a number of different settings JNK activation can be either pro- or anti-apoptotic. JNK activation is correlated with enhanced apoptosis in cardiac tissues following ischemia and reperfusion (Pombo C M, Bonventre J V, Avruch J, Woodgett J R, Kyriakis J. M, Force T. J. Biol. Chem. 269:26546-26551, 1994).

The JNK pathway leading to AP-1 appears to play a critical role in cancer. Expression of c-jun is altered in early lung cancer and may mediate growth factor signaling in non-small cell lung cancer (Yin T., Sandhu G., Wolfgang C. D., Burrier A., Webb R. L., Rigel D. F. Hai T., and Whelan J. J. Biol. Chem. 272:19943-19950, 1997). Indeed, over-expression of c-jun in cells results in transformation, and blocking c-jun activity inhibits MCF-7 colony formation (Szabo E., Riffe M., Steinberg S. M., Birrer M. J., Linnoila R. I. Cancer Res. 56:305-315, 1996). DNA-damaging agents, ionizing radiation and tumor necrosis factor activate the JNK pathway. In addition to regulating c-jun production and activity, JNK activation can regulate phosphorylation of p53, and thus can modulate cell cycle progression (Chen T. K., Smith L. M., Gebhardt D. K., Birrer M. J., Brown P. H. Mol. Carcinogenesis 15:215-226, 1996). The oncogene BCR-Abl, associated with t(9,22) Philadelphia chromosome translocation of chronic myelogenous leukemia, activates JNK and leads to transformation of hematopoietic cells (Milne D. M., Campbell L. E., Campbell D. G., Meek D. W. J. Biol. Chem. 270:5511-5518, 1995). Selective inhibition of JNK activation by a naturally occurring JNK inhibitory protein, called JIP-1, blocks cellular transformation caused by BCR-Abl expression (Raitano A. B., Halpern J. R., Hambuch T. M., Sawyers C. L. Proc. Nat. Acad. Sci USA 92:11746-11750, 1995). Thus, JNK inhibitors may block transformation and tumor cell growth.

The involvement of JNK in insulin mediated diseases such as Type II diabetes and obesity has also been confirmed (Hirosumi, J. et al. Nature 420:333-336, 2002). Elevated TNF-α expression in adipose tissue has also been linked to obesity and insulin resistance (Spiegelman, B. M. et al. J. Biol. Chem. 286(10):6823-6826, 1993). Additional studies have demonstrated that inhibition of the JNK pathway inhibits TNF-α lipolysis which has been implicated in diseases characterized by insulin resistance (International Publication No. WO 99/53927).

Selective and non-selective JNK inhibitors are being developed by a number of groups given the potential utility in the treatment of disease. One class of JNK inhibitors is indazoles. An example within this class is 1-(5-(1H-1,2,4-triazol-5-yl)(1H-indazol-3-yl))-3-(2-piperidylethoxy)benzene, which has the following structure:

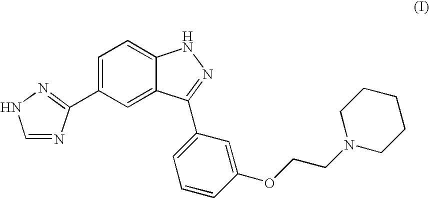

Compound (I) is disclosed in U.S. Pat. No. 6,897,231 B2, issued May 24, 2005, and in International Publication WO 02/10137, published Feb. 7, 2002.

Solid forms such as salts and crystal forms, e.g., polymorphic forms, of a compound are known in the pharmaceutical art to affect, for example, the solubility, stability, flowability, fractability, and compressibility of the compound as well as the safety and efficacy of drug products based on the compound, (see, e.g., Knapman, K. Modern Drug Discoveries, 2000:53). So critical are the potential effects of solid forms in a single drug product on the safety and efficacy of the respective drug product that the United States Food and Drug Administration requires the identification and control of solid forms, e.g., polymorphic forms, of each compound used in each drug product marketed in the United States. Accordingly, new solid forms of JNK inhibitors can further the development of formulations for the treatment of these diseases. The present invention provides such novel solid forms such as forms of the JNK inhibitor 1-(5-(1H-1,2,4-triazol-5-yl)(1H-indazol-3-yl))-3-(2-piperidylethoxy)benzene (referred to herein as Compound (I)).

### 3. SUMMARY OF THE INVENTION

The present invention provides novel solid forms including novel crystal forms of Compound (I) (referred to herein as “solid form(s) of the invention”) which are useful for the manufacture of a pharmaceutical product and for use in the treatment or prevention of a number of diseases, including, but not limited to liver disease, cancer, cardiovascular diseases, metabolic diseases, renal diseases, autoimmune conditions, inflammatory conditions, fibrotic diseases, macular degeneration, pain and related syndromes, disease-related wasting, asbestos-related conditions, pulmonary hypertension, ischemia/reperfusion injury or central nervous system (CNS) injury/damage. The solid forms of the invention are also useful for inhibiting JNK in a cell by contacting the cell with an effective amount of a solid form of the invention and treating or preventing a disease treatable or preventable by inhibiting JNK, comprising administering an effective amount of a solid form of the invention to a patient in need thereof.

The storage stability, compressibility, density or dissolution properties of the solid forms of the invention are beneficial for manufacturing, formulation and bio-availability of Compound (I). In particular, the solid forms described herein are beneficial for inhibiting JNK in a cell and treating or preventing a disease treatable or preventable by inhibiting JNK in a patient in need of such treatment or prevention.

The solid forms of the invention include forms of Compound (I) (also referred to herein as the “compound of the invention”), which is described in U.S. Pat. No. 6,897,231 B2, issued May 24, 2005, e.g., at column 168 (Example 243), U.S. Publication No. 2002/0103229 A1, published Aug. 1, 2002, e.g., at page 95, paragraph 1145 (Example 243) and in International Publication WO 02/10137, published Feb. 7, 2002, e.g., at page 259, lines 11-19 (Example 272), the contents of each are hereby incorporated by reference in their entirety. Compound (I) has the following structure:

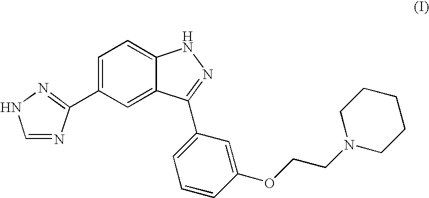

In certain aspects, the present invention provides crystalline solid forms of the invention identified as Forms A-J, each described in detail below. Each solid form is characterized by one or more physical properties, in particular, by x-ray powder diffraction patterns, infrared spectra and crystal lattice. Melting points, solubility, thermogravimentric analysis, differential scanning calorimetry and hygroscopicity can also be used to characterize the solid forms of the invention.

The present invention also provides pharmaceutical compositions comprising an effective amount of a solid form of the invention and a pharmaceutically acceptable carrier, diluent or excipient. These pharmaceutical compositions are useful for the treatment or prevention of a disease such as, but not limited to, liver disease, cancer, cardiovascular diseases, metabolic diseases, renal diseases, autoimmune conditions, inflammatory conditions, fibrotic diseases, macular degeneration, pain and related syndromes, disease-related wasting, asbestos-related conditions, pulmonary hypertension, ischemia/reperfusion injury or central nervous system (CNS) injury/damage. These pharmaceutical compositions are also useful for the treatment or prevention of a disease, condition or disorder mediated by JNK, such as a disease, condition or disorder that is treatable or preventable by the inhibition of JNK.

The present invention further provides methods for the treatment or prevention of a disease such as, but not limited to, liver disease, cancer, cardiovascular diseases, metabolic diseases, renal diseases, autoimmune conditions, inflammatory conditions, fibrotic diseases, macular degeneration, pain and related syndromes, disease-related wasting, asbestos-related conditions, pulmonary hypertension, ischemia/reperfusion injury or central nervous system (CNS) injury/damage comprising administering to a patient in need of such treatment or prevention an effective amount of a solid form of the invention.

The present invention also provides methods for the treatment or prevention of a disease, condition or disorder mediated by JNK, comprising administering to a patient in need of such treatment or prevention an effective amount of a solid form of the invention.

The present invention also provides methods for the inhibition of JNK in a cell comprising contacting the cell with an effective amount of a solid form of the invention.

In further embodiments, the present invention provides methods for making, isolating and/or characterizing the solid forms of the invention.

### 4. BRIEF DESCRIPTION OF THE DRAWINGS

FIG. 1 provides an XRPD diffractogram of Form A.

FIG. 2 provides an image and particle size analysis of Form A.

FIG. 3 provides a TGA plot of Form A.

FIG. 4 provides a DSC plot of Form A.

FIG. 5 provides a TGA plot of Form A after heating at 80° C. to remove residual solvents.

FIG. 6 provides a DSC plot of Form A after heating at 80° C. to remove residual solvents.

FIG. 7 provides a DVS plot of form A.

FIG. 8 provides an XRPD diffractogram of Form B.

FIG. 9 provides an image and particle size analysis of Form B.

FIG. 10 provides a TGA plot of Form B.

FIG. 11 provides a DSC plot of Form B.

FIG. 12 provides a DVS plot of form B.

FIG. 13 provides an XRPD diffractogram of Form C.

FIG. 14 provides an image and particle size analysis of Form C.

FIG. 15 provides a TGA plot of Form C.

FIG. 16 provides a DSC plot of Form C.

FIG. 17 provides a DVS plot of form C.

FIG. 18 provides an XRPD diffractogram of Form D.

FIG. 19 provides an image and particle size analysis of Form D.

FIG. 20 provides a TGA plot of Form D.

FIG. 21 provides a DSC plot of Form D.

FIG. 22 provides a DVS plot of form D.

FIG. 23 provides an XRPD diffractogram of Form E.

FIG. 24 provides an image and particle size analysis of Form E.

FIG. 25 provides a TGA plot of Form E.

FIG. 26 provides a DSC plot of Form E.

FIG. 27 provides a DVS plot of form E.

FIG. 28 provides an XRPD diffractogram of Form F.

FIG. 29 provides an image and particle size analysis of Form F.

FIG. 30 provides a TGA plot of Form F.

FIG. 31 provides a DSC plot of Form F.

FIG. 32 provides a DVS plot of form F.

FIG. 33 provides an XRPD diffractogram of Form G.

FIG. 34 provides an image and particle size analysis of Form G.

FIG. 35 provides a TGA plot of Form G.

FIG. 36 provides a DSC plot of Form G.

FIG. 37 provides a DVS plot of form G.

FIG. 38 provides an XRPD diffractogram of Form H.

FIG. 39 provides an image and particle size analysis of Form H.

FIG. 40 provides a TGA plot of Form H.

FIG. 41 provides a DSC plot of Form H.

FIG. 42 provides a DVS plot of form H.

FIG. 43 provides an XRPD diffractogram of Form I.

FIG. 44 provides an image and particle size analysis of Form I.

FIG. 45 provides a TGA plot of Form I.

FIG. 46 provides a DSC plot of Form I.

FIG. 47 provides a DVS plot of form I.

FIG. 48 provides an XRPD diffractogram of Form J.

FIG. 49 provides an image and particle size analysis of Form J.

FIG. 50 provides a TGA plot of Form J.

FIG. 51 provides a DSC plot of Form J.

FIG. 52 provides a DVS plot of form J.

### 5. DETAILED DESCRIPTION OF THE INVENTION

5.1 Definitions

A “patient” is defined herein to include an animal (e.g., cow, horse, sheep, pig, chicken, turkey, quail, cat, dog, mouse, rat, rabbit or guinea pig), in one embodiment a mammal such as a non-primate or a primate (e.g., monkey or human), and in another embodiment a human. In certain embodiments, the patient is an infant, child, adolescent or adult.

The term “JNK” means a protein, or an isoform thereof, expressed by a JNK 1, JNK 2, or JNK 3 gene (Gupta, S., Barrett, T., Whitmarsh, A. J., Cavanagh, J., Sluss, H. K., Derijard, B. and Davis, R. J. The EMBO J. 15:2760-2770, 1996).

The term “effective amount” means the amount of a solid form of the invention that will elicit the biological or medical response of a tissue, system, animal or human that is being sought by the researcher, veterinarian, medical doctor or other clinician, or that is sufficient to prevent development of or alleviate to some extent one or more of the symptoms of the disease being treated.

The terms “treat”, “treating” or “treatment”, as used herein, refer to an amelioration of a disease or disorder, or at least one discernible symptom thereof. In another embodiment, “treatment” or “treating” refer to inhibiting the progression of a disease or disorder, either physically, e.g., stabilization of a discernible symptom, physiologically, e.g., stabilization of a physical parameter, or both.

The terms “prevent”, “preventing” or “prevention”, as used herein, refer to a reduction of the risk of acquiring a given disease or disorder. For example, a solid form of the compound of the invention can be administered to a patient who is at risk for having a particular disease or disorder due to, for example, genetic or environmental factors.

The terms, “polymorphs” and “polymorphic forms” and related terms herein refer to solid forms of the compound of the invention having different physical properties as a result of the order of the molecules in the crystal lattice. The differences in physical properties exhibited by solid forms affect pharmaceutical parameters such as storage stability, compressibility and density (important in formulation and product manufacturing), and dissolution rates (an important factor in determining bioavailability). Differences in stability can result from changes in chemical reactivity (e.g., differential oxidation, such that a dosage form discolors more rapidly when comprised of one solid form than when comprised of another solid form) or mechanical changes (e.g., tablets crumble on storage as a kinetically favored polymorph converts to thermodynamically more stable solid form) or both (e.g., tablets of one solid form are more susceptible to breakdown at high humidity). As a result of solubility/dissolution differences, in the extreme case, some solid form transitions may result in lack of potency or, at the other extreme, toxicity. In addition, the physical properties of the crystal may be important in processing, for example, one solid form might be more likely to form solvates or might be difficult to filter and wash free of impurities (i.e., particle shape and size distribution might be different between one solid form relative to the other).

As used herein, a solid form that is “pure,” i.e., substantially free of other solid forms, contains less than about 10% of one or more other solid forms, less than about 5% of one or more other solid forms, less than about 3% of one or more other solid forms, less than about 1% of one or more other solid forms, or less than about 0.1% of one or more other solid forms as determined by one skilled in the art, e.g., using x-ray powder diffraction or infrared spectrometry. The purity of a solid form can be determined by XRPD.

Solid forms of a molecule can be obtained by a number of methods, such as those known in the art. Such methods include, but are not limited to, melt recrystallization, melt cooling, solvent recrystallization, desolvation, rapid evaporation, rapid cooling, slow cooling, vapor diffusion and sublimation. Polymorphism can be detected, or identified, and classified using e.g., X-ray powder diffractometry (“XRPD”), differential scanning calorimetry (“DSC”), thermogravimetry (“TGA”), single crystal X-ray diffractometry, vibrational spectroscopy, solution calorimetry, solid state NMR, IR spectroscopy, Raman spectroscopy, hot stage optical microscopy, scanning electron microscopy (“SEM”), electron crystallography and quantitative analysis, particle size analysis (“PSA”), surface area analysis, solubility, rate of dissolution and hygroscopicity.

The term “pharmaceutically acceptable salt(s)” is meant to include salts of the solid forms of the invention which are prepared with relatively nontoxic acids. Acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, et al. (1977) J. Pharm. Sci. 66:1-19).

The neutral forms of the compounds can be regenerated by contacting the salt with a base or acid and isolating the parent solid form in the conventional manner. The parent solid form of the compound differs from the various salt forms in certain physical properties, such as solubility in polar solvents, but otherwise the salts are equivalent to the parent form of the compound for the purposes of the present invention.

By “pharmaceutically acceptable,” it is meant that the carrier, diluent or excipient must be compatible with the other ingredients of the formulation and not deleterious to the recipient thereof.

5.2 Embodiments of the Invention

The present invention is directed to solid forms of the compound of the invention, compositions comprising the solid forms and methods for their use in the treatment or prevention of disease and/or the inhibition of JNK. The storage stability, compressibility, density or dissolution properties of the solid forms are beneficial for manufacturing, formulation and bio-availability of the compound of the invention.

Preferred solid forms of the present invention are those which inhibit at least one function or characteristic of a mammalian JNK protein, for example, a human JNK protein. In one embodiment, the JNK protein is JNK1, JNK2 or JNK3. The ability of a solid form to inhibit such a function can be demonstrated by any JNK inhibition assay known in the art, such as that disclosed herein in Example 13. Exemplary assays are described in U.S. Publication No. 2002/0103229 A1, published Aug. 1, 2002, and International Publication WO 02/10137, published Feb. 7, 2002, the contents of each of which are hereby incorporated by reference in their entirety.

5.2.1 Solid Forms

The present invention provides solid forms of Compound (I), a JNK inhibitor, having particular utility for the treatment or prevention of liver disease, cancer, cardiovascular diseases, metabolic diseases, renal diseases, autoimmune conditions, inflammatory conditions, fibrotic diseases, macular degeneration, pain and related syndromes, disease-related wasting, asbestos-related conditions, pulmonary hypertension, ischemia/reperfusion injury or central nervous system (CNS) injury/damage or a disease treatable or preventable by inhibiting JNK. Compound (I) has the following structure:

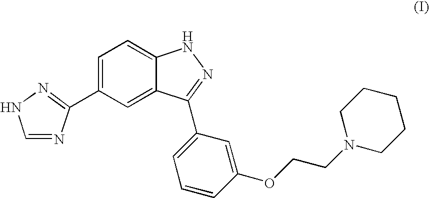

Each solid form of the invention can be made, as described below in section 5.2.2, from a preparation of Compound (I). Compound (I) can be synthesized or obtained according to any method apparent to one skilled in the art. In one embodiment, Compound (I) is prepared according to the methods described in detail in the examples below as well as in U.S. Publication No. 2002/0103229 A1, published Aug. 1, 2002, and International Publication WO 02/10137, published Feb. 7, 2002.

An exemplary scheme for the synthesis of Compound (I) is described in detail in Example 1, below. An exemplary scheme for the large-scale synthesis of Compound (I) is described in detail in Example 2, below.

5.2.2 Preparation and Characterization of Solid Forms

### 5.2.2.1 Form A

In one embodiment, the present invention provides Form A as a crystal form of the invention.

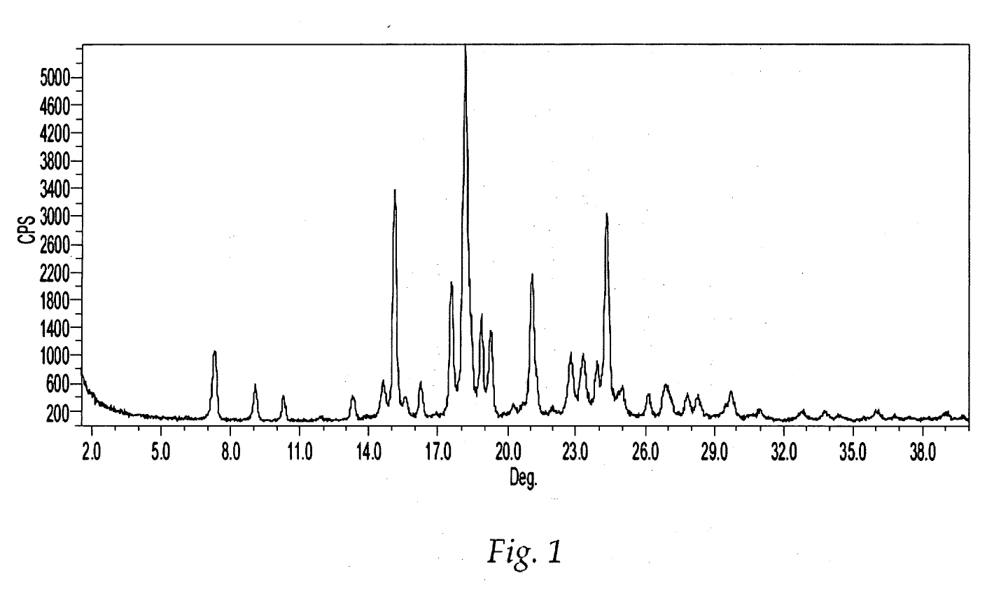

Form A can be made by any method apparent to those of skill in the art to obtain a polymorph with an XRPD diffractogram having characteristic peaks at 7.3, 15.2, 17.7, 18.3, 21.2 and 24.5 (see FIG. 1) or their substantial equivalent. In one embodiment, Form A can be obtained by recrystallization of Compound (I) from acetonitrile. In another embodiment, Form A can be obtained by equilibrating Compound (I) in heptane, methylene chloride or water.

In a further embodiment, Form A can be obtained by THF-acetonitrile solvent exchange, wherein THF is gradually replaced by acetonitrile. In one embodiment, a solution comprising amorphous Compound (I) in THF is slowly distilled followed by gradual addition of acetonitrile. In a particular embodiment, the THF is distilled off at a reduced pressure of including, but not limited to, about 300 to about 600 torr, or about 320 to about 600 torr. In another embodiment, the THF is distilled off at a temperature of including, but not limited to, about 40° C. to about 55° C., or about 40° C. to about 50° C. In another embodiment, Form A can be prepared from amorphous Compound 1 at a scale of up to about 10 g, up to about 50 g or up to about 100 g in a yield of about 75%, about 80%, about 85% or about 90%. In one embodiment, the solvent exchange process uses about 5 volumes of THF and about 15-20 volumes of acetonitrile.

In one embodiment, Form A melts at about 135° C. to about 140° C. In another embodiment, Form A melts at about 138° C. to about 140° C. In another embodiment, Form A melts at about 140° C.

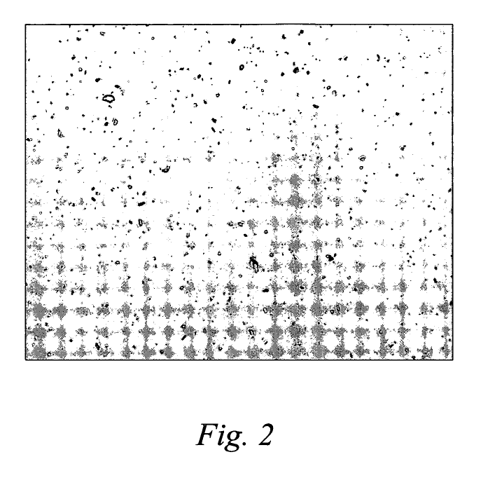

991 In another embodiment, Form A is a white, flaky crystalline solid with a particle size D₉₀<8 μm (see FIG. 2).

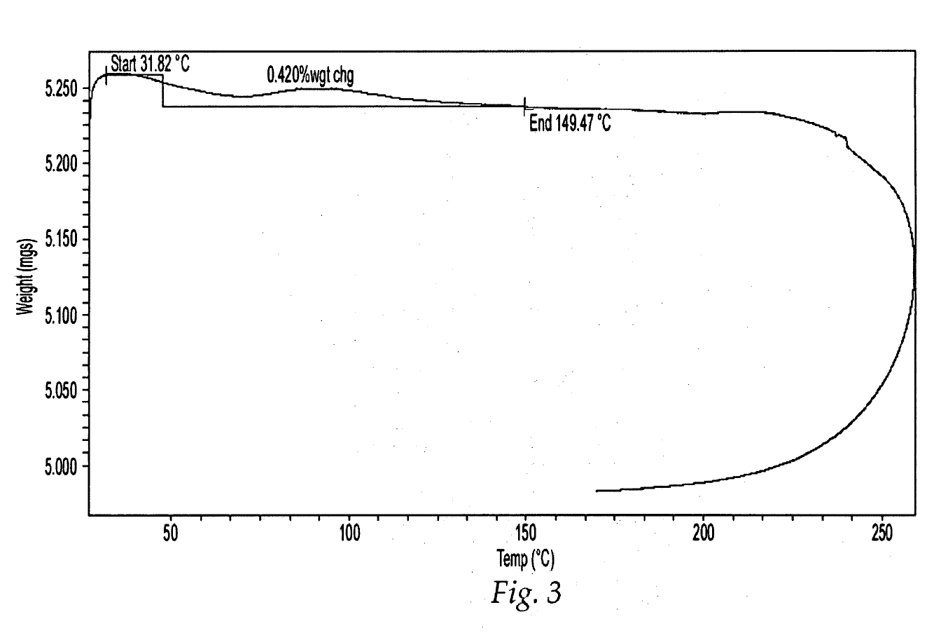

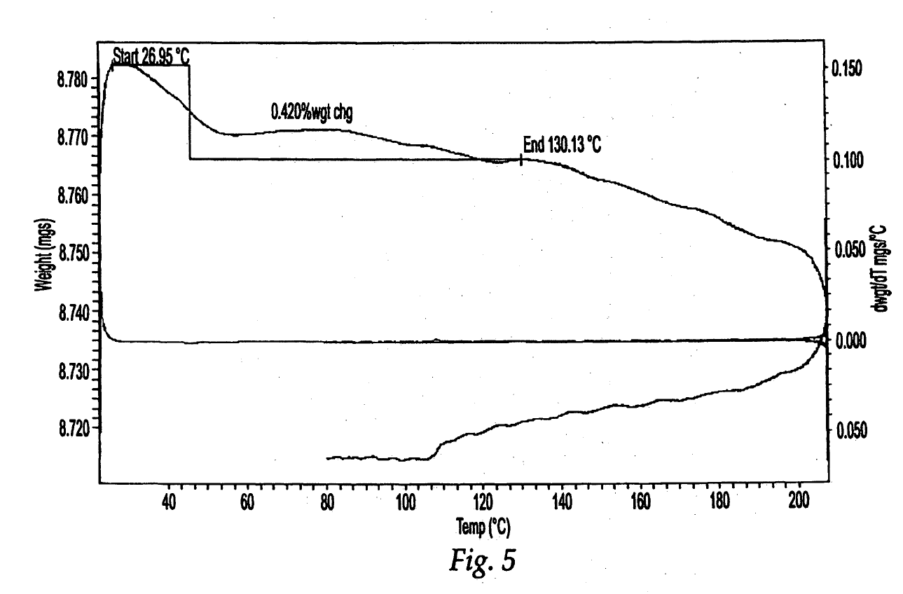

In another embodiment, Form A loses up to about 0.4% volatiles up to 150° C. by TGA (see FIG. 3) and up to about 0.2% volatiles up to 150° C. by TGA after heating at 80° C. to remove volatile solvent (see FIG. 5).

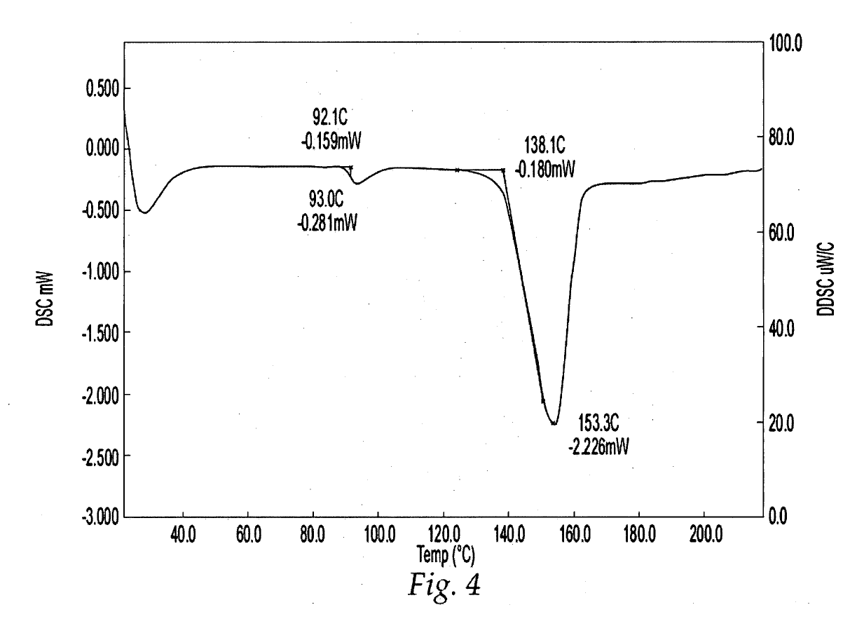

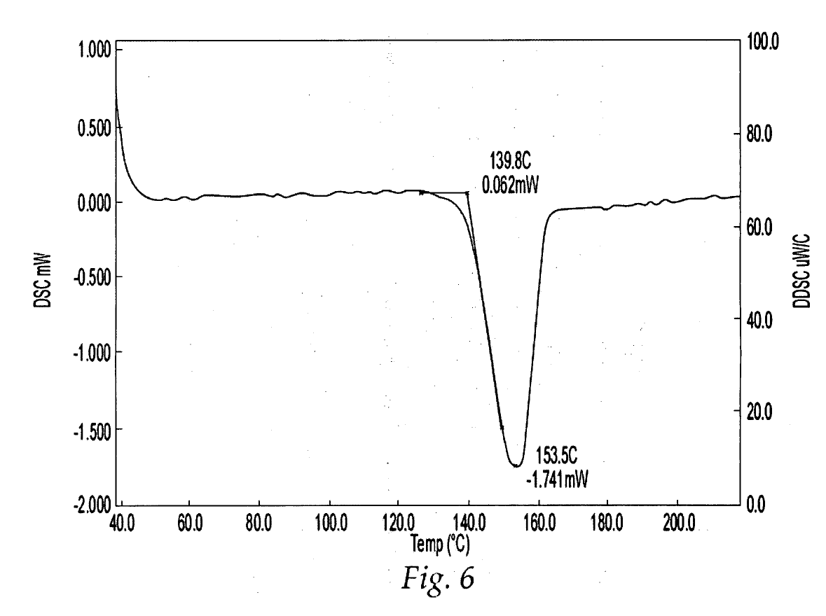

In another embodiment, Form A shows endothermal events at 92° C. and 138° C., and a melting temperature maximum of about 153° C. (see FIG. 4), wherein the endothermal event at 92° C. is eliminated upon heating at 80° C. to remove volatile solvent (see FIG. 6).

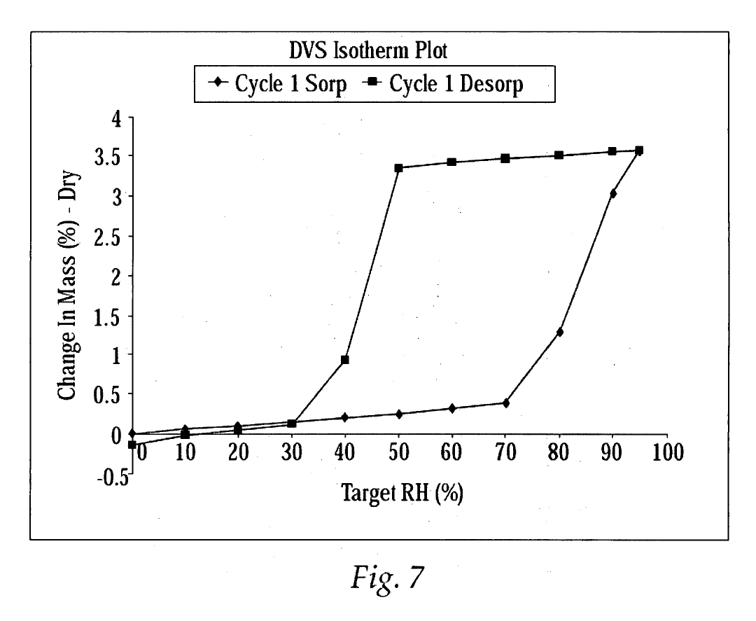

In another embodiment, Form A is not hygroscopic at 25° C. below 75% relative humidity (see FIG. 7).

In another embodiment, the XRPD diffractogram of Form A is unchanged after undergoing a full adsorption/desorption cycle.

In another embodiment, the XRPD diffractogram of Form A is unchanged after exposure to a 40° C./75% relative humidity environment for four weeks.

In another embodiment, Form A is stable in acetonitrile and in water.

In another embodiment, the XRPD diffractogram of Form A is unchanged after the application of 2000 psi pressure for one minute.

In another embodiment, Form A can be converted to Form B, C, D, E, F, G, H, I or J by equilibration in acetone, 2-propanol, n-butyl acetate, toluene, methyl t-butyl ether, ethyl acetate, tetrahydrofuran, ethanol or toluene, respectively.

### 5.2.2.2 Form B

In one embodiment, the present invention provides Form B as a crystal form of the invention.

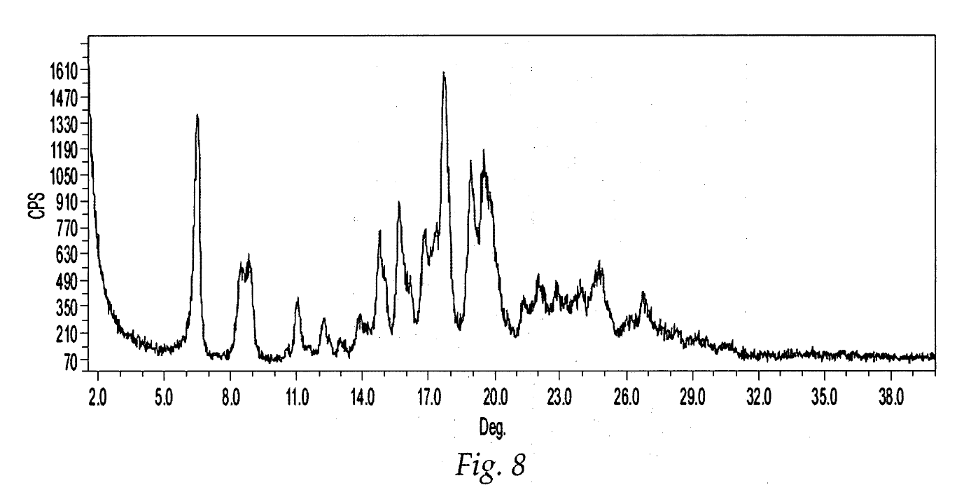

Form B can be made by any method apparent to those of skill in the art to obtain a polymorph with an XRPD diffractogram having characteristic peaks at 6.5, 8.5, 8.9, 14.9, 15.9, 18.0, 19.0, 19.6 and 24.9 (see FIG. 8) or their substantial equivalent. In one embodiment, Form B can be obtained by equilibration of Form A, prepared as described above, in acetone. In another embodiment, Form B can be obtained by recrystallization of Form A from acetone.

In one embodiment, Form B melts at about 135° C. to about 140° C. In another embodiment, Form B melts at about 137° C. to about 140° C. In another embodiment, Form B melts at about 137° C.

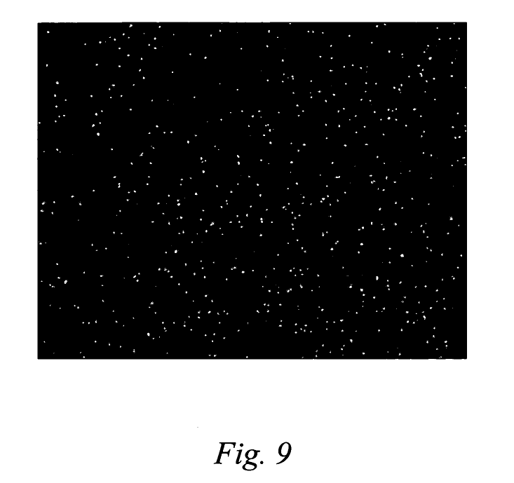

In another embodiment, Form B is a white, flaky crystalline solid with a particle size D₉₀<6 μm (see FIG. 9).

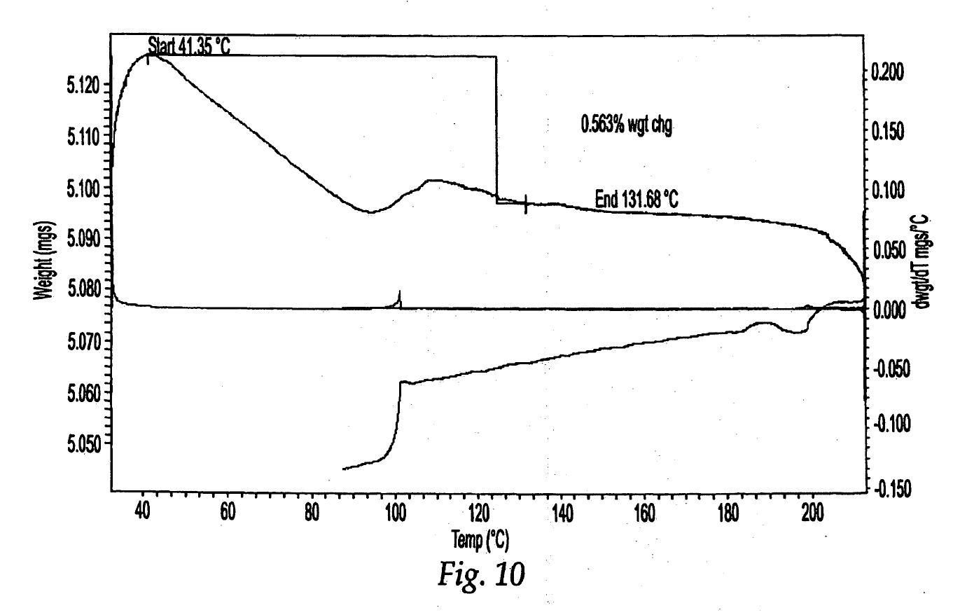

In another embodiment, Form B loses up to about 0.6% volatiles up to 130° C. by TGA (see FIG. 10).

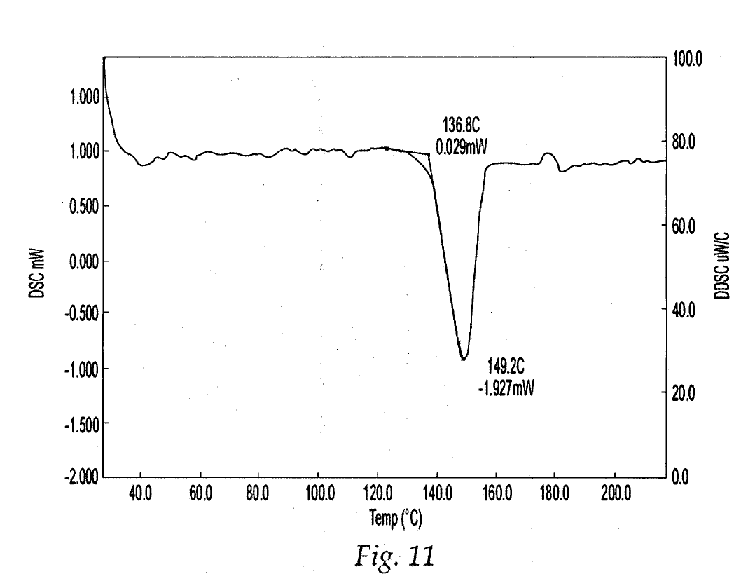

In another embodiment, Form B shows a single endothermal event at 137° C., and a melting temperature maximum of about 149° C. (see FIG. 11).

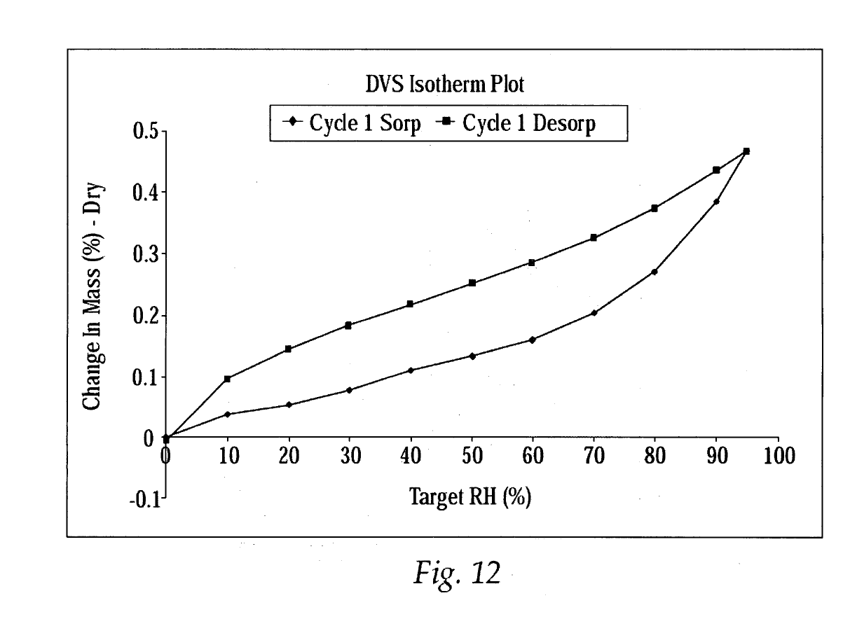

In another embodiment, Form B is not hygroscopic at 25° C. below 90% relative humidity (see FIG. 12).

In another embodiment, the XRPD diffractogram of Form B is unchanged after undergoing a full adsorption/desorption cycle.

In another embodiment, Form B can be converted to Form A by equilibrating in acetonitrile.

In another embodiment, Form B can be converted to Form H and amorphous material by exposure to a 40° C./75% relative humidity environment for about four weeks.

### 5.2.2.3 Form C

In one embodiment, the present invention provides Form C as a crystal form of the invention.

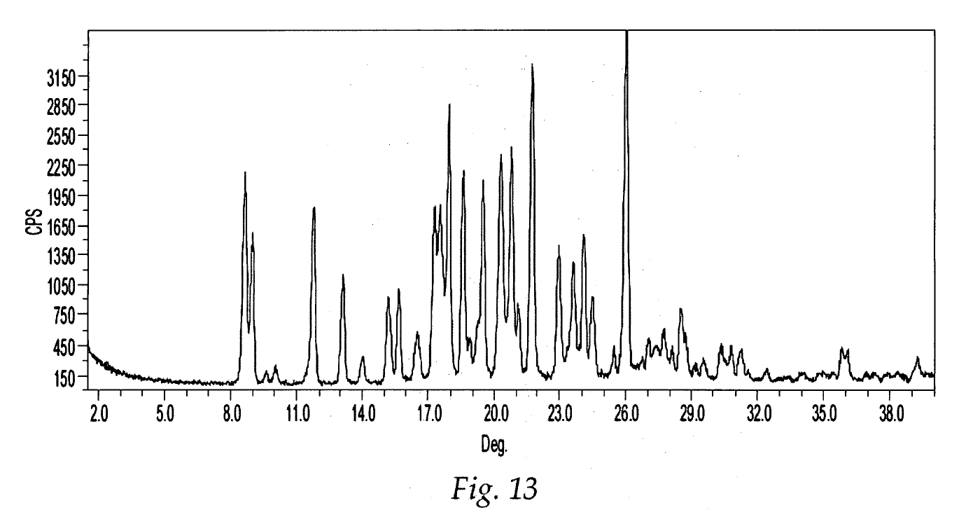

Form C can be made by any method apparent to those of skill in the art to obtain a polymorph with an XRPD diffractogram having characteristic peaks at 8.6, 11.8, 18.0, 21.9 and 26.0 (see FIG. 13) or their substantial equivalent. In one embodiment, Form B can be obtained by equilibration of Compound (I) in 2-propanol followed by evaporation of the solvent. In another embodiment, Form C can be obtained by recrystallization of Form A from 2-propanol.

In one embodiment, Form C melts at about 105° C. to about 110° C. In another embodiment, Form C melts at about 108° C. to about 110° C. In another embodiment, Form C melts at about 110° C.

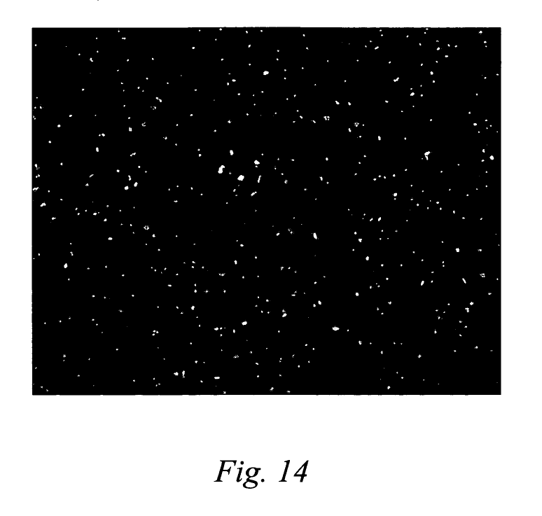

In another embodiment, Form C is a white, plated crystalline solid with a particle size D₉₀<12 μm (see FIG. 14).

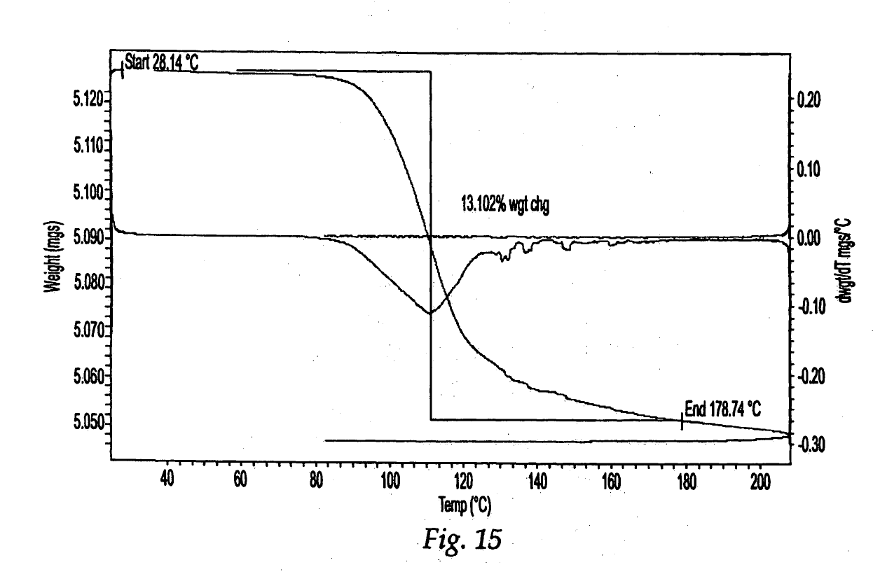

In another embodiment, Form C is obtained in a molar ratio of 1:1 of Compound (I) and 2-propanol as evidenced by TGA (see FIG. 15).

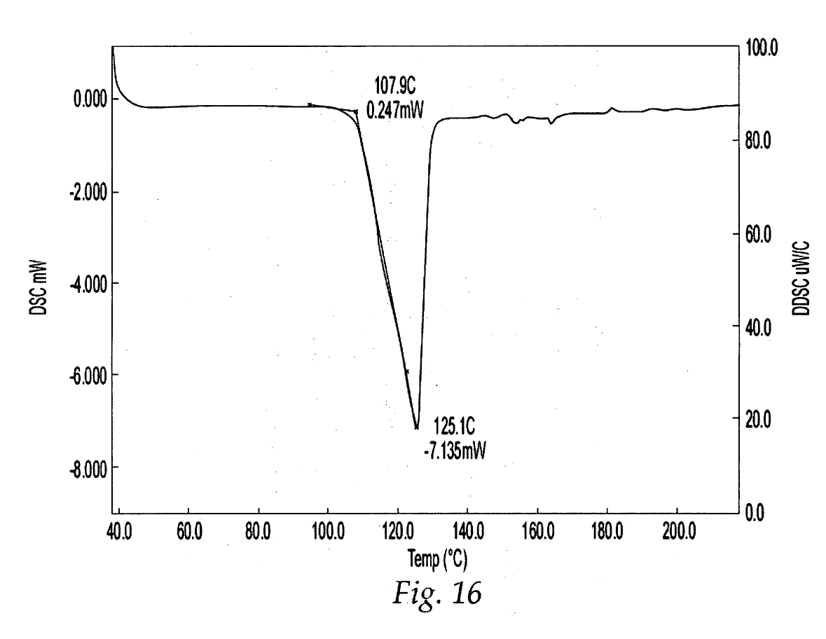

In another embodiment, Form C shows a single endothermal event at 108° C., and a melting temperature maximum of about 125° C. (see FIG. 16).

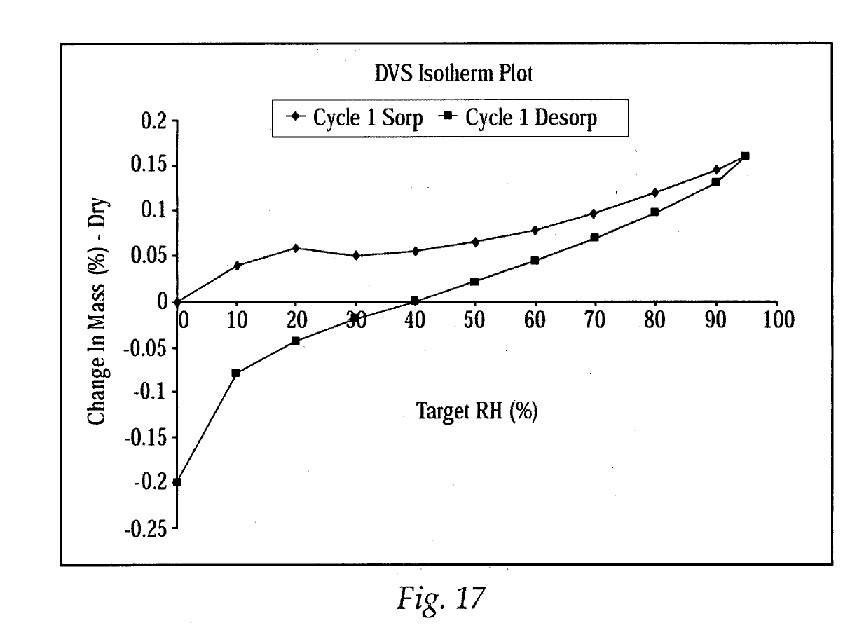

In another embodiment, Form C is not hygroscopic at 25° C. below 90% relative humidity (see FIG. 17).

In another embodiment, the XRPD diffractogram of Form C is unchanged after undergoing a full adsorption/desorption cycle.

In another embodiment, Form C can be converted to Form A by equilibrating in acetonitrile.

In another embodiment, Form C can be converted to partially amorphous material by exposure to a 40° C./75% relative humidity environment for about four weeks.

### 5.2.2.4 Form D

In one embodiment, the present invention provides Form D as a crystal form of the invention.

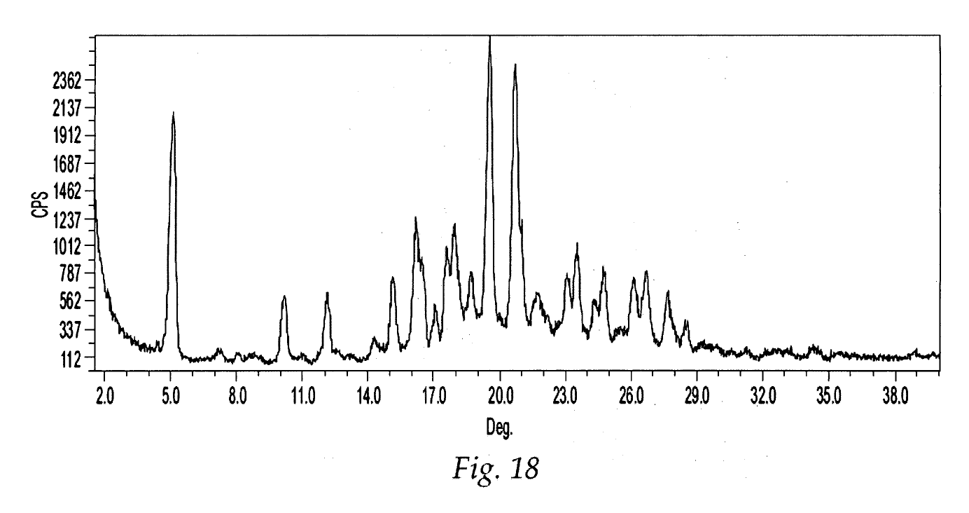

Form D can be made by any method apparent to those of skill in the art to obtain a polymorph with an XRPD diffractogram having characteristic peaks at 5.0, 10.2, 12.2, 15.2, 16.2, 18.0, 19.6, 20.9 and 23.7 (see FIG. 18) or their substantial equivalent. In one embodiment, Form D can be obtained by equilibration of Form A, prepared as described above, in n-butyl acetate.

In one embodiment, Form D melts at about 135° C. to about 140° C. In another embodiment, Form D melts at about 138° C. to about 140° C. In another embodiment, Form D melts at about 138° C.

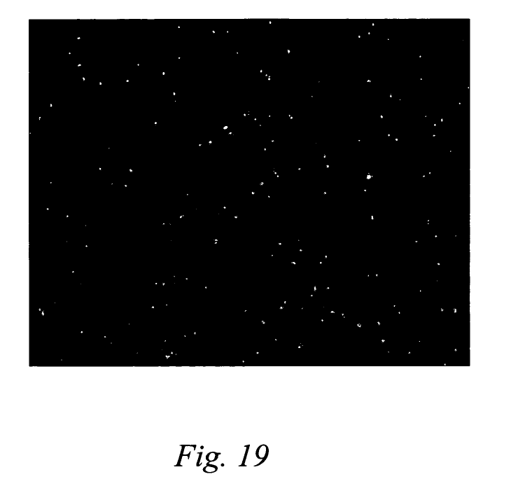

In another embodiment, Form D is a white, flaky crystalline solid with a particle size D₉₀<6 μm (see FIG. 19).

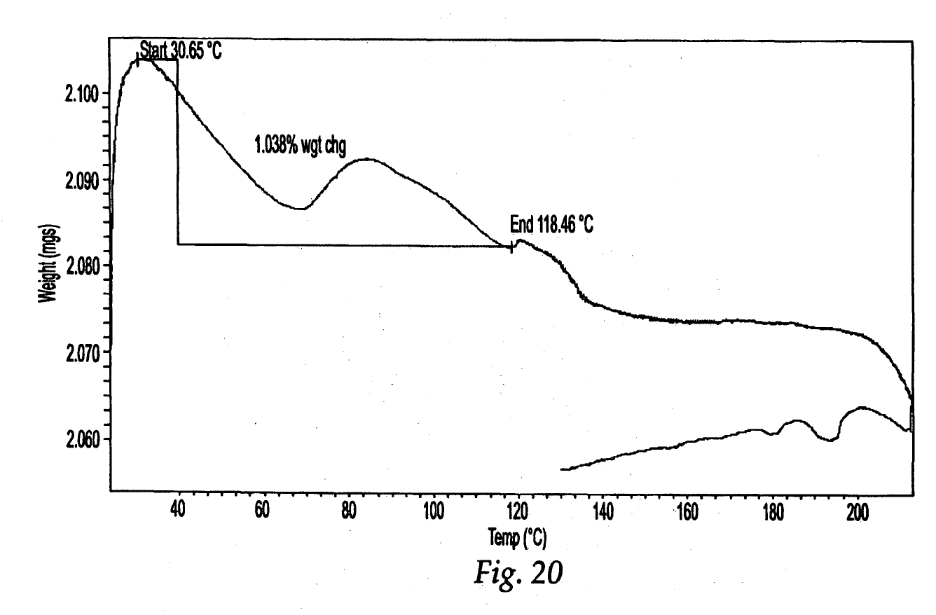

In another embodiment, Form D loses up to about 1.0% volatiles up to 120° C. by TGA (see FIG. 20).

In another embodiment, Form D is unsolvated.

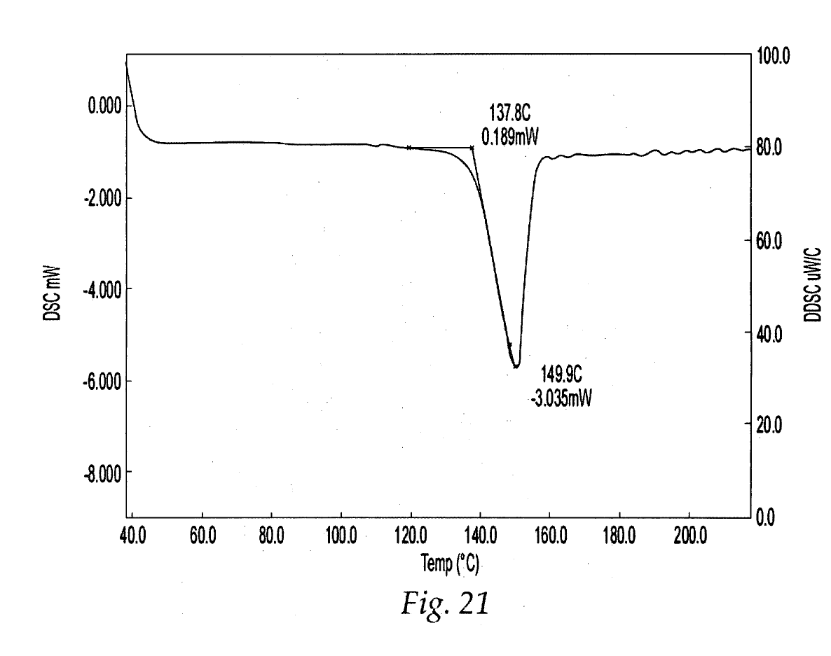

In another embodiment, Form D shows a single endothermal event at 138° C., and a melting temperature maximum of about 150° C. (see FIG. 21).

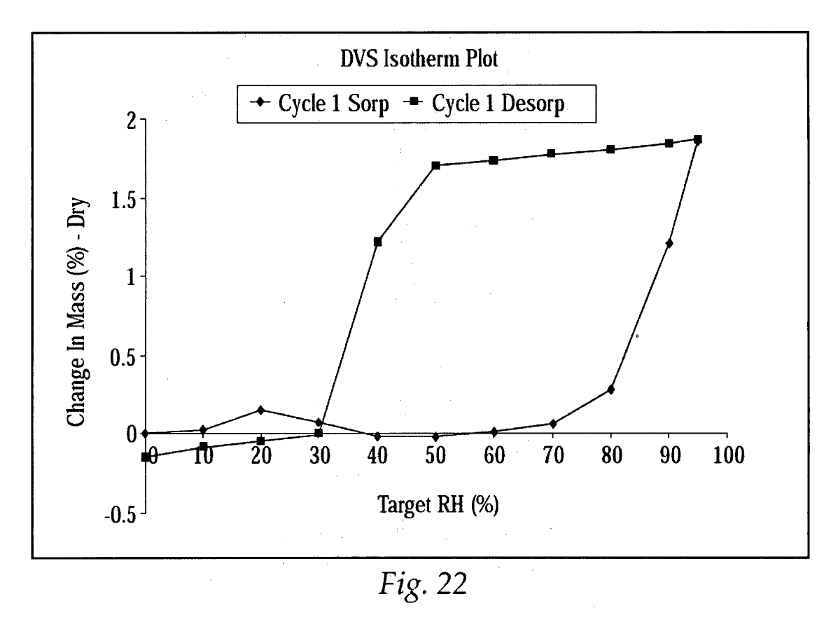

In another embodiment, Form D is not hygroscopic at 25° C. below 70% relative humidity (see FIG. 22). In another embodiment, Form D is hygroscopic at 25° C. between 70-90% relative humidity (see FIG. 22).

In another embodiment, Form D can be converted to Form A by equilibrating in acetonitrile.

In another embodiment, Form D can be converted to Form A by heating to 80° C.

In another embodiment, Form D can be converted to Form A after undergoing a full adsorption/desorption cycle.

In another embodiment, Form D is partially converted to Form A after storage in ambient conditions for about 50 days.

### 5.2.2.5 Form E

In one embodiment, the present invention provides Form E as a crystal form of the invention.

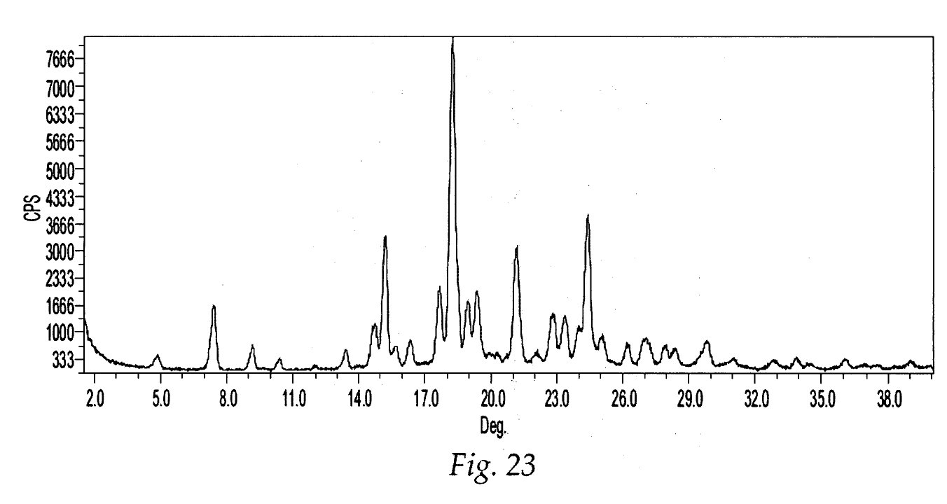

Form E can be made by any method apparent to those of skill in the art to obtain a polymorph with an XRPD diffractogram having characteristic peaks at 7.4, 15.3, 18.3, 21.2 and 24.5 (see FIG. 23) or their substantial equivalent. In one embodiment, Form E can be obtained by equilibration of Form A, prepared as described above, in toluene at 25° C.

In one embodiment, Form E melts at about 135° C. to about 140° C. In another embodiment, Form E melts at about 137° C. to about 140° C. In another embodiment, Form E melts at about 137° C.

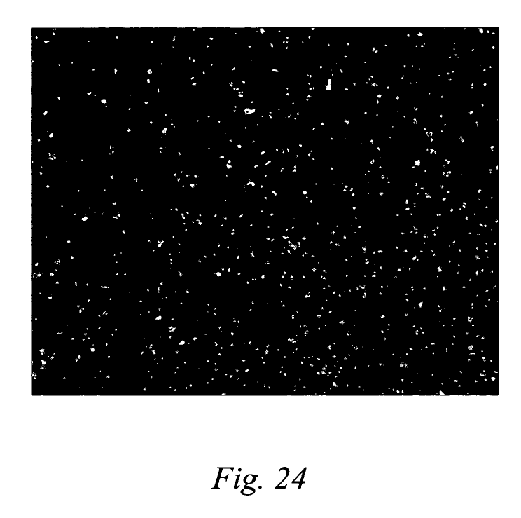

In another embodiment, Form E is a white, flaky crystalline solid with a particle size D₉₀<6 μm (see FIG. 24).

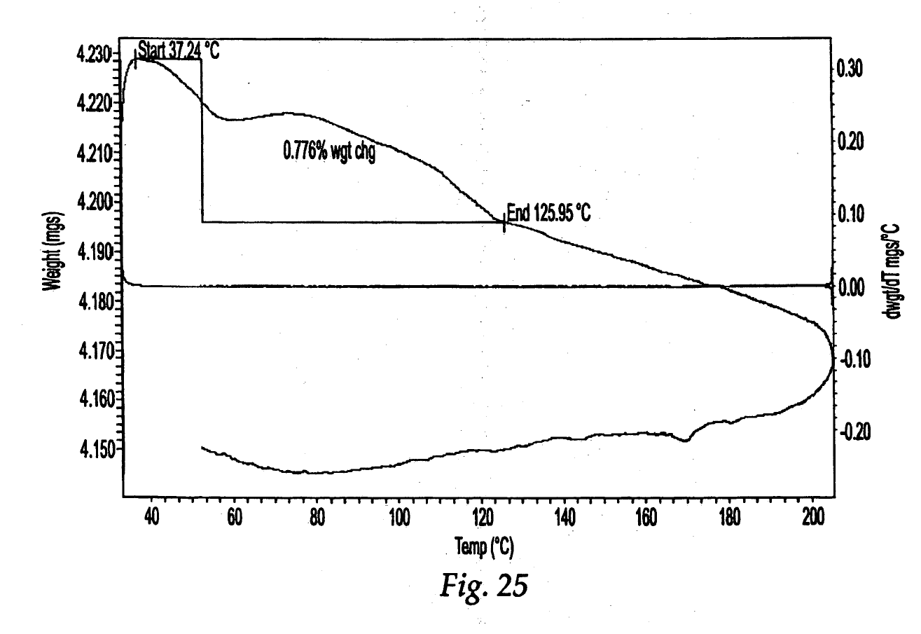

In another embodiment, Form E loses up to about 0.8% volatiles up to 125° C. by TGA (see FIG. 25).

In another embodiment, Form E is unsolvated.

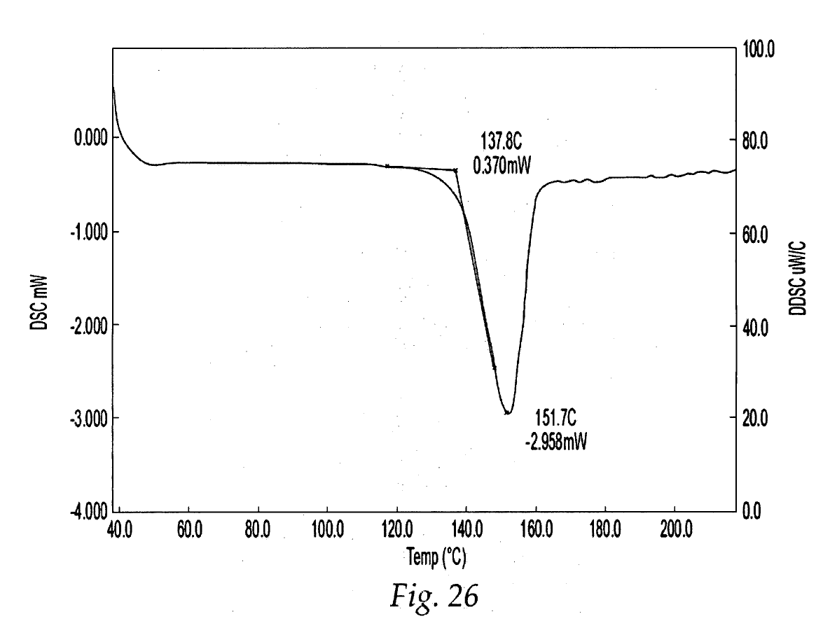

In another embodiment, Form E shows a single endothermal event at 137° C., and a melting temperature maximum of about 152° C. (see FIG. 26).

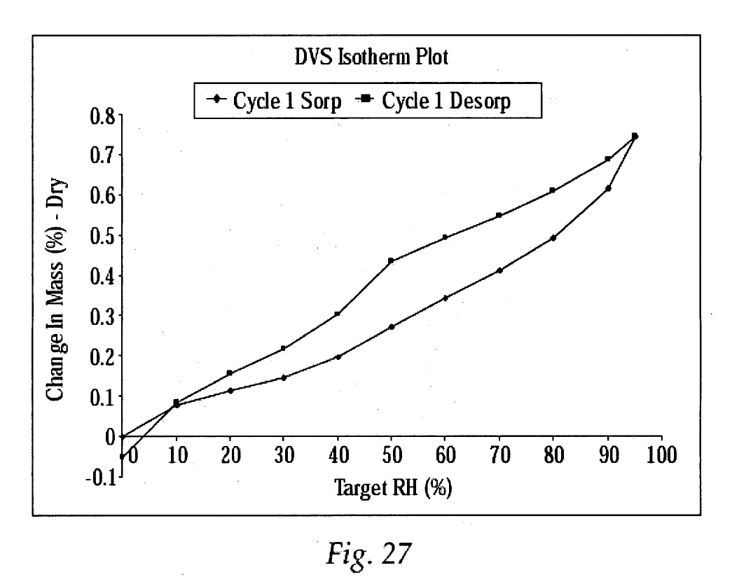

In another embodiment, Form E is not hygroscopic at 25° C. below 90% relative humidity (see FIG. 27).

In another embodiment, the XRPD diffractogram of Form E is unchanged after undergoing a full adsorption/desorption cycle.

In another embodiment, Form E can be converted to Form A by equilibrating in acetonitrile.

In another embodiment, Form E can be partially converted to amorphous material by exposure to a 40° C./75% relative humidity environment for about four weeks.

In another embodiment, Form E changes from a white solid to a yellow solid after exposure to a 40° C./75% relative humidity environment for about four weeks.

### 5.2.2.6 Form F

In one embodiment, the present invention provides Form F as a crystal form of the invention.

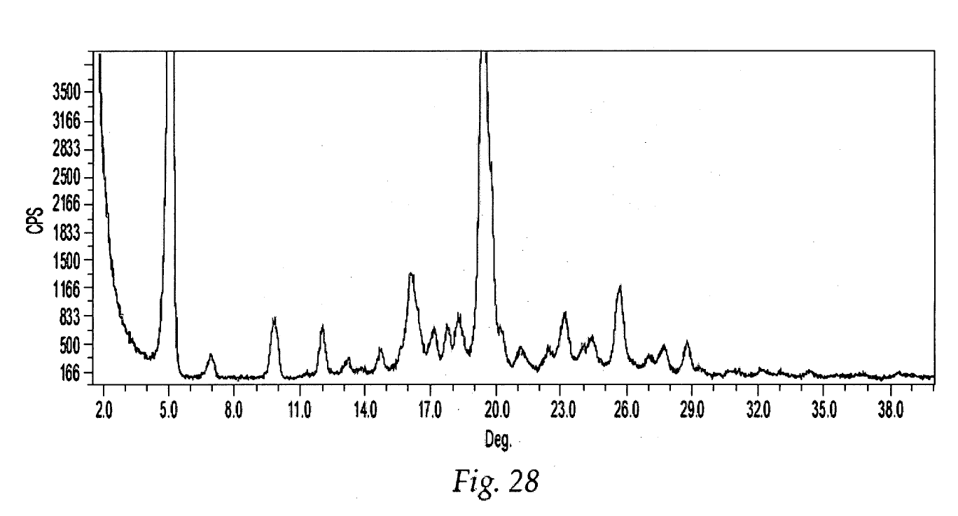

Form F can be made by any method apparent to those of skill in the art to obtain a polymorph with an XRPD diffractogram having characteristic peaks at 5.0, 9.9, 16.1, 19.7 and 25.8 (see FIG. 28) or their substantial equivalent. In one embodiment, Form F can be obtained by equilibration of Form A, prepared as described above, in methyl t-butyl ether and by recrystallization of Form A in methyl t-butyl ether.

In one embodiment, Form F begins to melt at about 120° C. to about 130° C. In another embodiment, Form F begins to melt at about 126° C. to about 130° C. In another embodiment, Form F begins to melt at about 126° C.

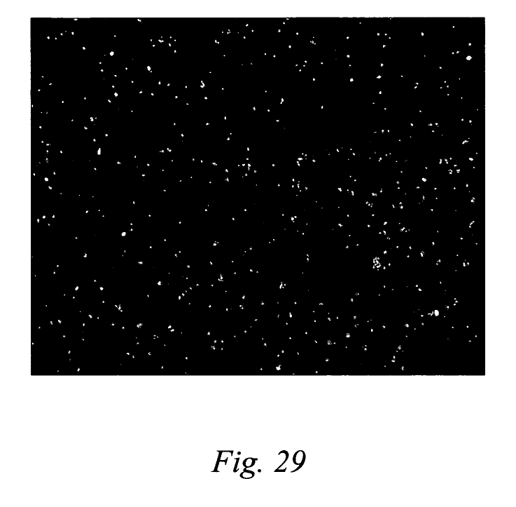

In another embodiment, Form F is a white, flaky crystalline solid with a particle size D₉₀<6 μm (see FIG. 29).

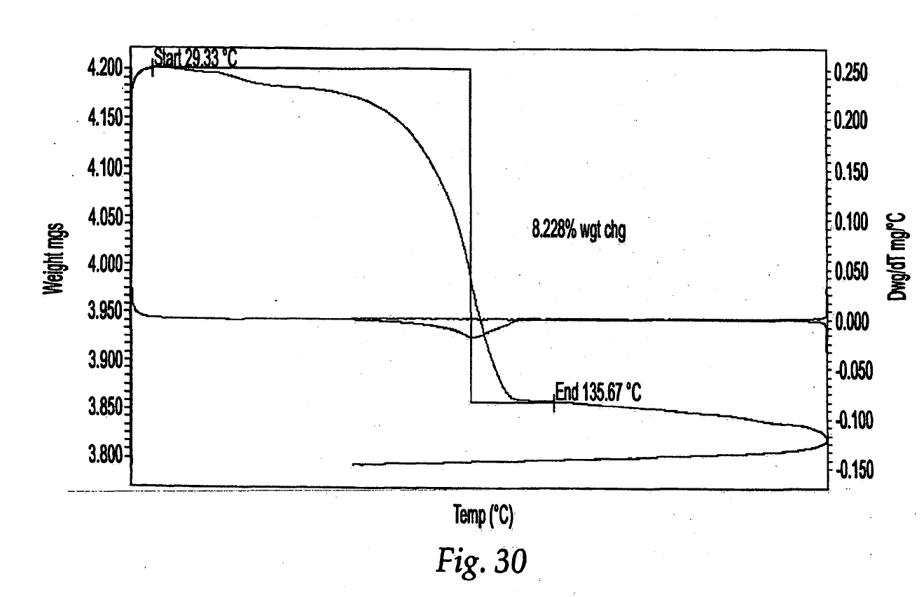

In another embodiment, Form F loses up to about 8-9% volatiles up to 135° C. by TGA (see FIG. 30).

In another embodiment, Form F is solvated.

In another embodiment, Form F is a mixture of polymorph forms.

In another embodiment, Form F is obtained in a molar ratio of 2.5:1 of Compound (I) and methyl t-butyl ether as evidenced by TGA (see FIG. 30).

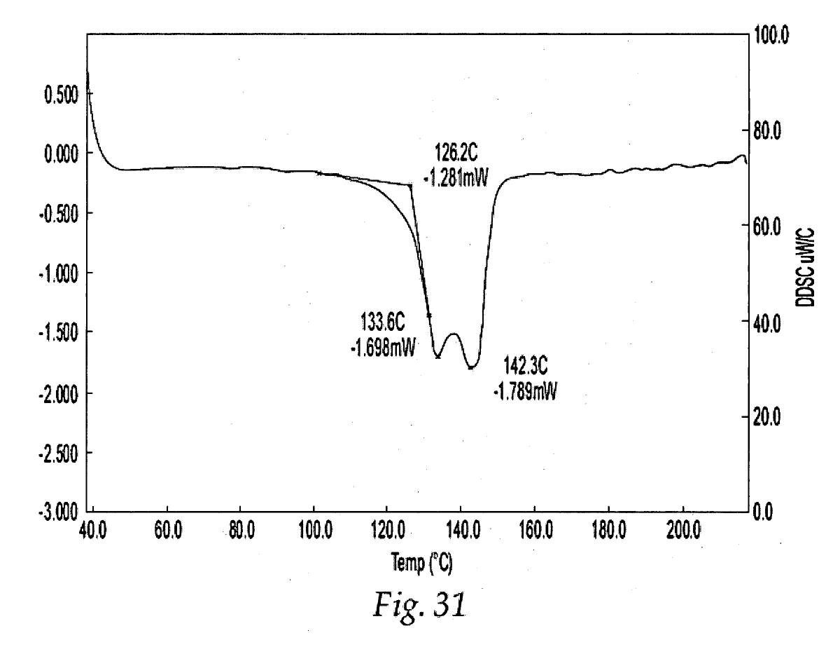

In another embodiment, Form F shows a broad doublet endothermal event beginning at 126° C., and a melting temperature maximum of about 142° C. (see FIG. 31).

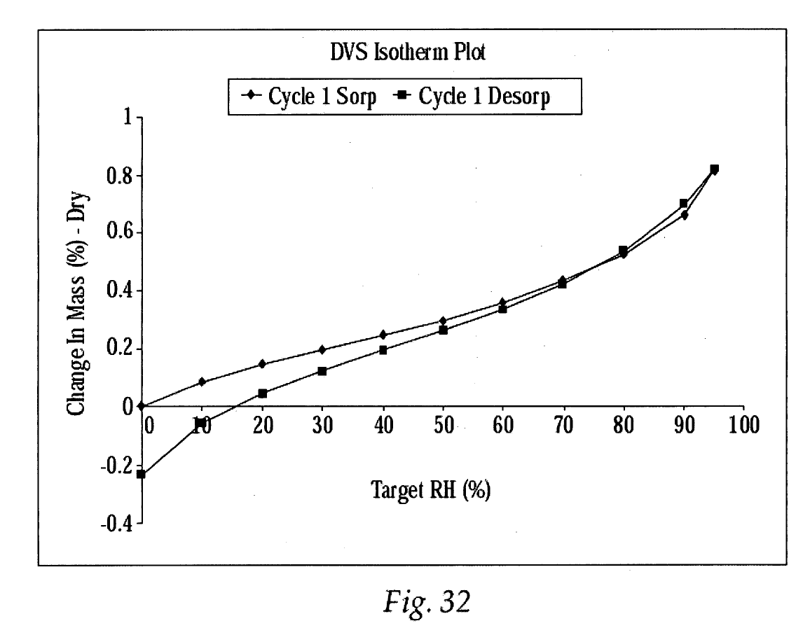

In another embodiment, Form F is not hygroscopic at 25° C. below 95% relative humidity (see FIG. 32).

In another embodiment, the XRPD diffractogram of Form F is unchanged after undergoing a full adsorption/desorption cycle.

In another embodiment, Form F can be converted to Form A by equilibrating in acetonitrile.

### 5.2.2.7 Form G

In one embodiment, the present invention provides Form G as a crystal form of the invention.

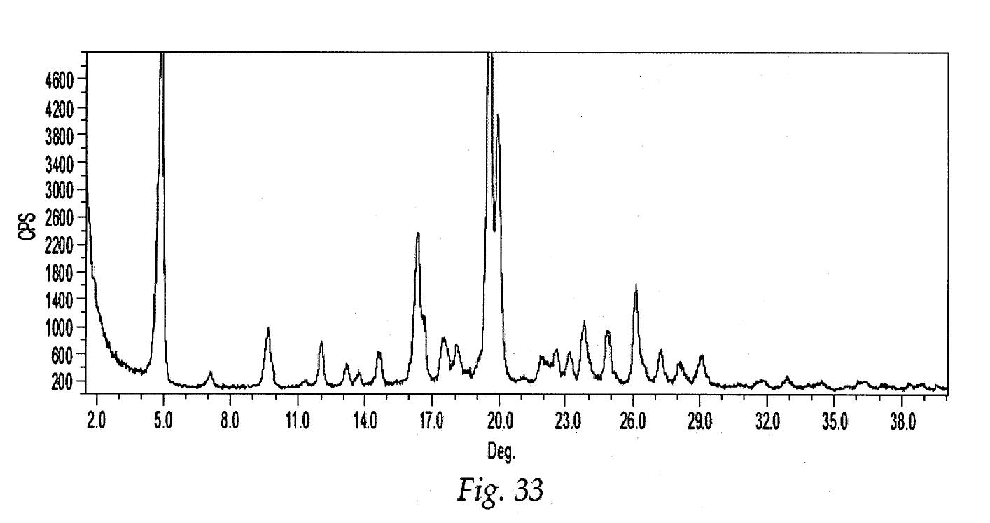

Form G can be made by any method apparent to those of skill in the art to obtain a polymorph with an XRPD diffractogram having characteristic peaks at 4.9, 9.7, 16.4, 19.8, 20.0 and 26.2 (see FIG. 33) or their substantial equivalent. In one embodiment, Form G can be obtained by evaporation of a solution of Form A, prepared as described above, in methyl ethyl ketone. In another embodiment, Form G can be obtained by slurrying Form A in methyl ethyl ketone. In another embodiment, Form G can be obtained by slurrying Form A in ethyl acetate. In another embodiment, Form G can be obtained by recrystallization of Form A from methyl ethyl ketone. In another embodiment, Form G can be obtained by precipitation of Form A from ethanol by the addition of heptane as an anti-solvent. In another embodiment, Form G can be obtained by precipitation of Form A from tetrahydrofuran by the addition of heptane as an anti-solvent.

In one embodiment, Form G melts at about 130° C. to about 140° C. In another embodiment, Form G melts at about 134° C. to about 140° C. In another embodiment, Form G melts at about 134° C.

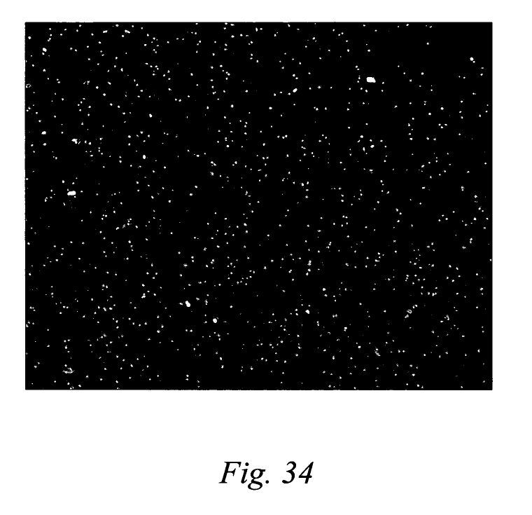

In another embodiment, Form G is a white, flaky crystalline solid with a particle size D₉₀<6 μm (see FIG. 34).

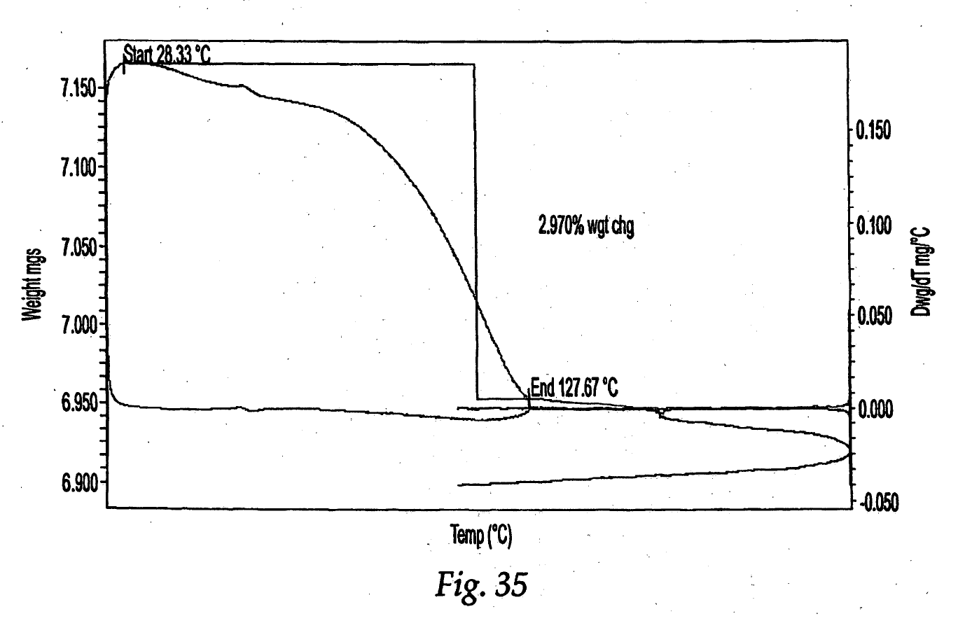

In another embodiment, Form G loses up to about 3.0% volatiles up to 130° C. by TGA (see FIG. 35).

In another embodiment, Form G is solvated.

In another embodiment, Form G is obtained in a molar ratio of 7:1 of Compound (I) and methyl ethyl ketone as evidenced by TGA (see FIG. 35).

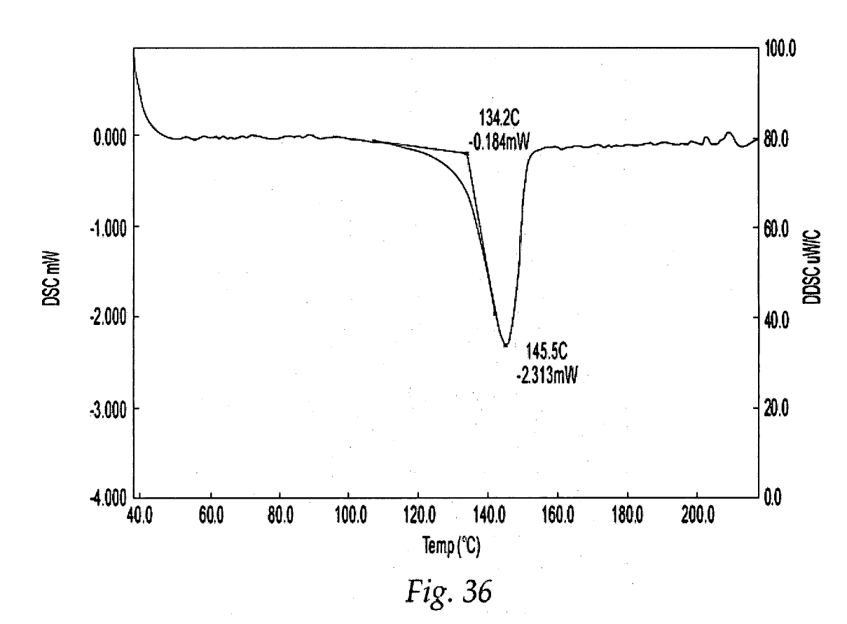

In another embodiment, Form G is a mixture of polymorphs and shows a broad multiplet of endothermal events. In another embodiment, Form G obtained by recrystallization of Form A from methyl ethyl ketone shows a single endothermal event at 134° C., and a melting temperature maximum of about 146° C. (see FIG. 36).

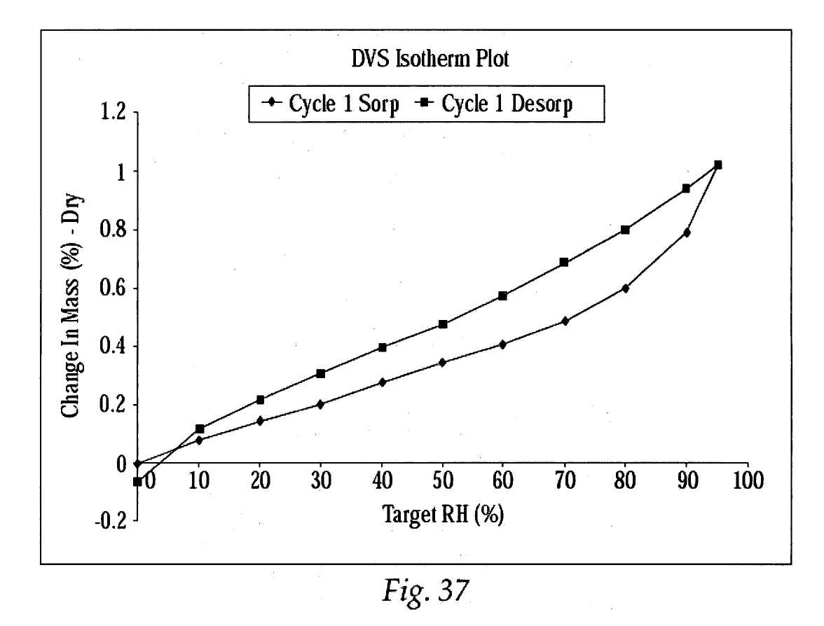

In another embodiment, Form G is only slightly hygroscopic (i.e., exhibits an increase of about 1% in mass relative to the dry mass) at 25° C. up to 95% relative humidity (see FIG. 37).

In another embodiment, the XRPD diffractogram of Form G is unchanged after undergoing a full adsorption/desorption cycle.

In another embodiment, Form G can be converted to Form A by equilibrating in acetonitrile.

In another embodiment, the XRPD diffractogram of Form G is unchanged after exposure to a 40° C./75% relative humidity environment for about four weeks.

### 5.2.2.8 Form H

In one embodiment, the present invention provides Form H as a crystal form of the invention.

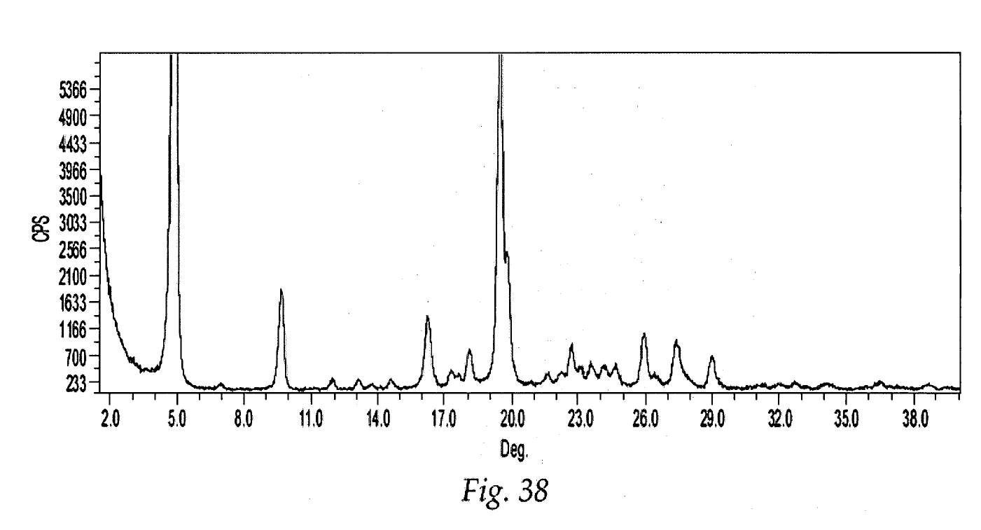

Form H can be made by any method apparent to those of skill in the art to obtain a polymorph with an XRPD diffractogram having characteristic peaks at 4.8, 9.7, 16.2, 19.6 and 26.0 (see FIG. 38) or their substantial equivalent. In one embodiment, Form H can be obtained by evaporation of a solution of Form A, prepared as described above, in tetrahydrofuran. In another embodiment, Form H can be obtained by slurrying Form A in tetrahydrofuran. In another embodiment, Form H can be obtained by recrystallization of Form A from tetrahydrofuran. In another embodiment, Form H can be obtained by precipitation of Form A from tetrahydrofuran by the addition of water as an anti-solvent.

In one embodiment, Form H melts at about 115° C. to about 125° C. In another embodiment, Form H melts at about 119° C. to about 125° C. In another embodiment, Form H melts at about 119° C.

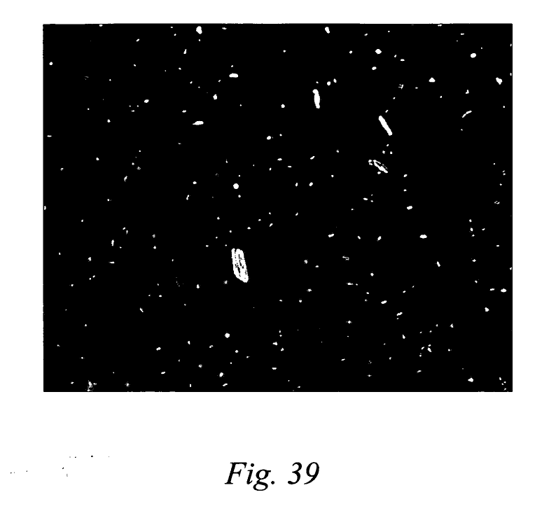

In another embodiment, Form H is a white, flaky crystalline solid with a particle size D₉₀<20 μm (see FIG. 39).

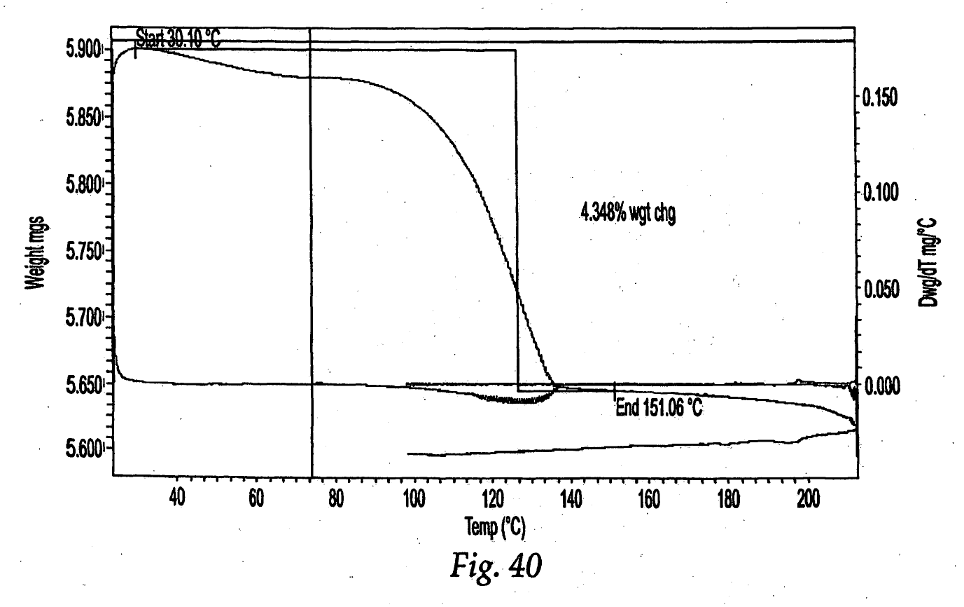

In another embodiment, Form H loses up to about 4.5% volatiles up to 130° C. by TGA (see FIG. 40).

In another embodiment, Form H is solvated.

In another embodiment, Form H is obtained in a molar ratio of 5:1 of Compound (I) and tetrahydrofuran as evidenced by TGA (see FIG. 40).

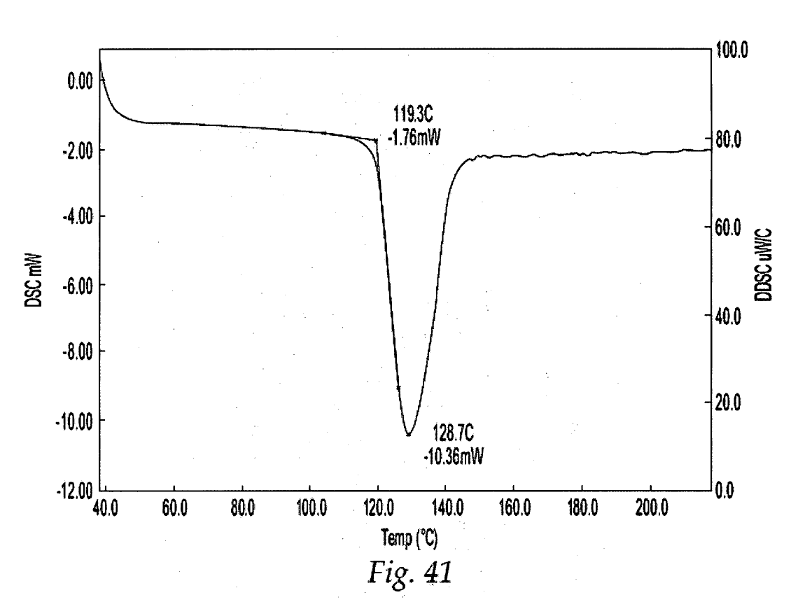

In another embodiment, Form H is a mixture of polymorphs. In another embodiment, Form H obtained by recrystallization of Form A from tetrahydrofuran shows a single endothermal event at 119° C., and a melting temperature maximum of about 129° C. (see FIG. 41).

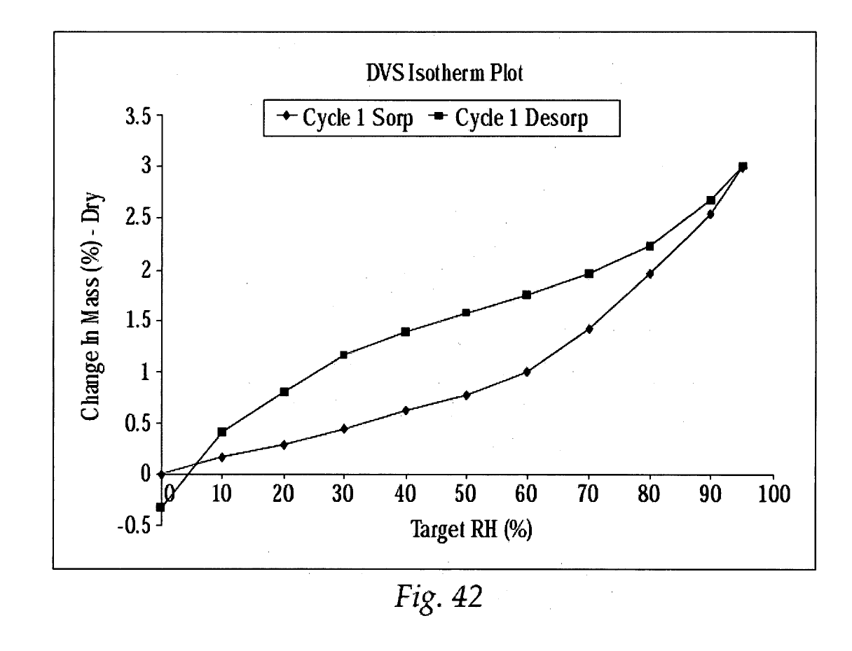

In another embodiment, Form H is hygroscopic (i.e., exhibits an increase of about 3% in mass relative to the dry mass) at 25° C. up to 95% relative humidity (see FIG. 42).

In another embodiment, the XRPD diffractogram of Form H is unchanged after undergoing a full adsorption/desorption cycle.

In another embodiment, Form H can be converted to Form A by equilibrating in acetonitrile.

In another embodiment, Form H can be partially converted to amorphous material by exposure to a 40° C./75% relative humidity environment for about four weeks.

In another embodiment, Form H changes from a white solid to a yellow solid after exposure to a 40° C./75% relative humidity environment for about four weeks.

### 5.2.2.9 Form I

In one embodiment, the present invention provides Form I as a crystal form of the invention.

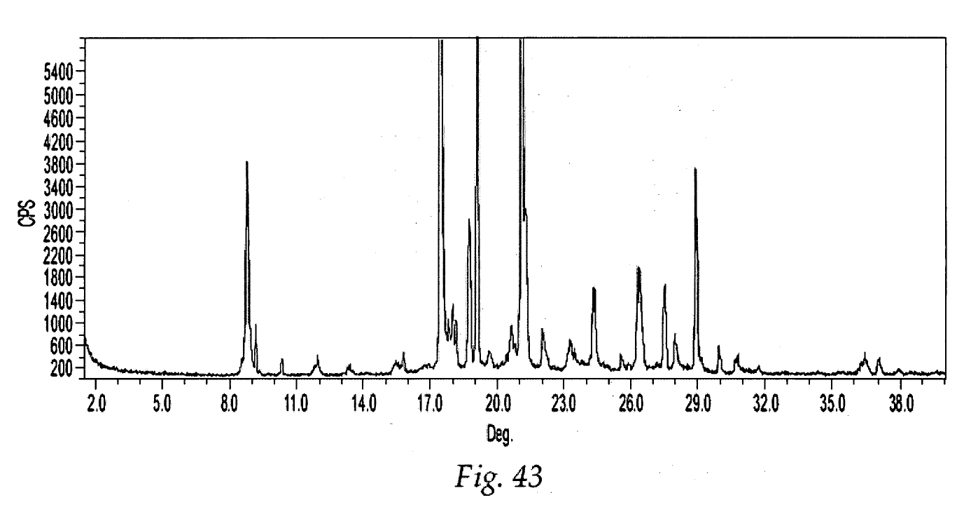

Form I can be made by any method apparent to those of skill in the art to obtain a polymorph with an XRPD diffractogram having characteristic peaks at 8.8, 17.6, 18.8, 19.2, 21.2, 24.3, 26.4 and 29.0 (see FIG. 43) or their substantial equivalent. In one embodiment, Form I can be obtained by evaporation of a solution of Form A, prepared as described above, in ethanol.

In one embodiment, Form I melts at about 95° C. to about 105° C. In another embodiment, Form I melts at about 98° C. to about 105° C. In another embodiment, Form I melts at about 98° C.

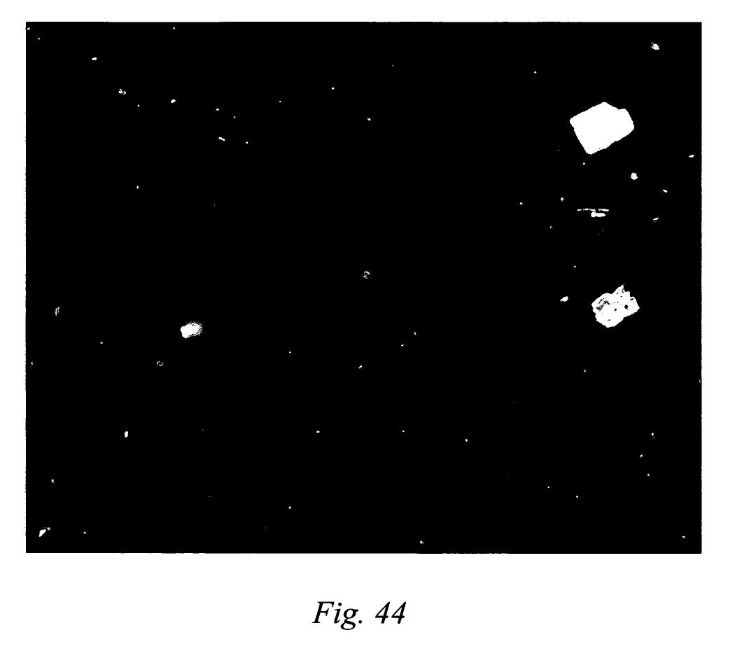

In another embodiment, Form I is a mixture of amorphous material and glass-like plate crystalline material (see FIG. 44).

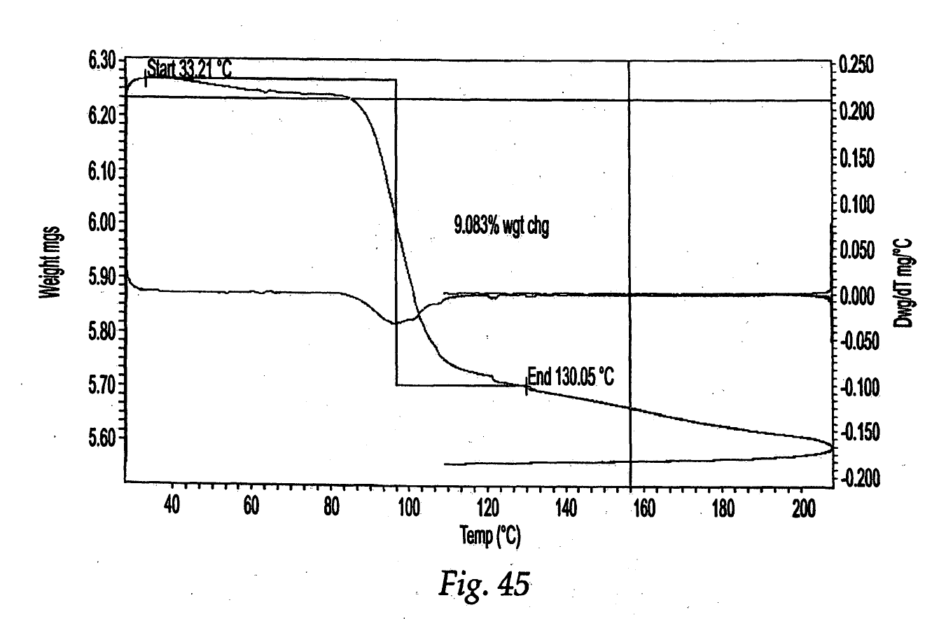

In another embodiment, Form I loses up to about 9.1% volatiles up to 130° C. by TGA (see FIG. 45).

In another embodiment, Form I is solvated.

In another embodiment, Form I is obtained in a molar ratio of 1:1 of Compound (I) and ethanol as evidenced by TGA (see FIG. 45).

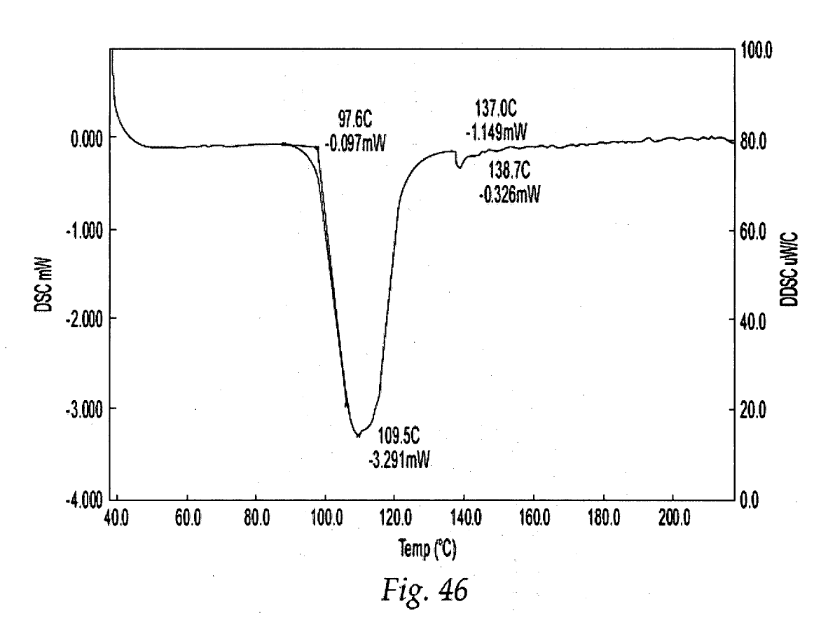

In another embodiment, Form I shows a single endothermal event at 98° C., and a melting temperature maximum of about 110° C. (see FIG. 46).

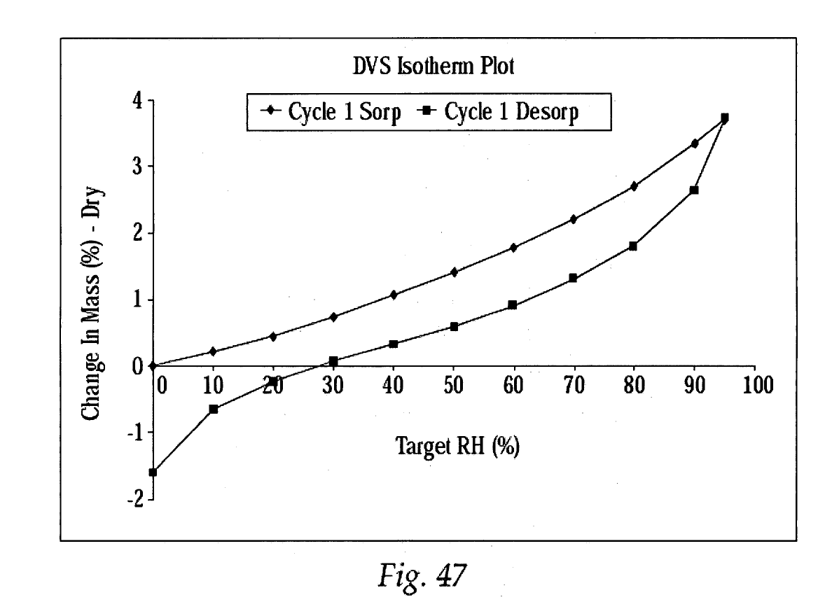

In another embodiment, Form I is hygroscopic (i.e., exhibits an increase of about 3.8% in mass relative to the dry mass) at 25° C. up to 95% relative humidity (see FIG. 47).

In another embodiment, the XRPD diffractogram of Form I is partially converted to an amorphous material after undergoing a full adsorption/desorption cycle.

In another embodiment, Form I can be converted to Form A by equilibrating in acetonitrile.

In another embodiment, Form I can be partially converted to amorphous material by exposure to a 40° C./75% relative humidity environment for about four weeks.

In another embodiment, Form I changes from a white solid to a yellow solid after exposure to a 40° C./75% relative humidity environment for about four weeks.

### 5.2.2.10 Form J

In one embodiment, the present invention provides Form J as a crystal form of the invention.

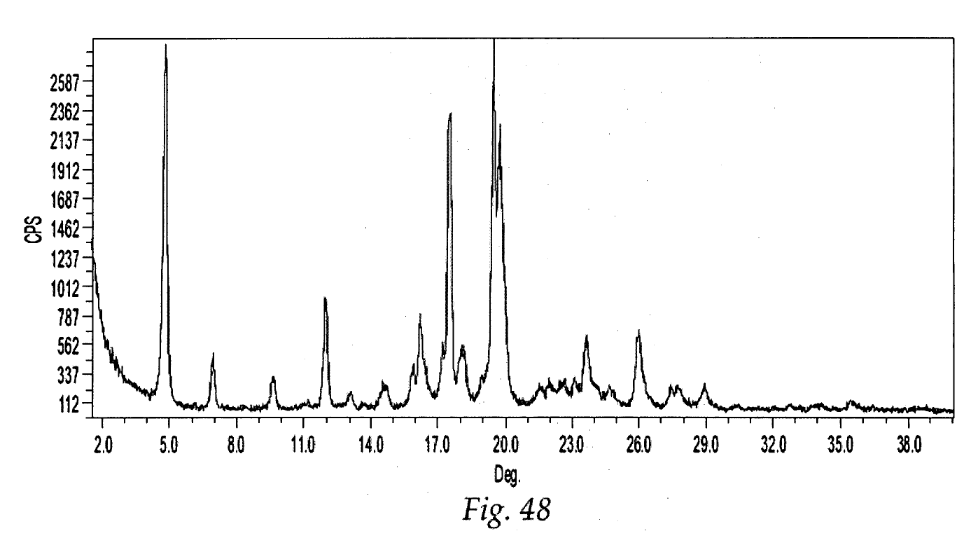

Form J can be made by any method apparent to those of skill in the art to obtain a polymorph with an XRPD diffractogram having characteristic peaks at 4.8, 12.0, 16.2, 17.6, 19.6, 20.0, 23.7 and 26.0 (see FIG. 48) or their substantial equivalent. In one embodiment, Form J can be obtained by precipitation of Form A from methyl ethyl ketone by the addition of heptane as an anti-solvent. In another embodiment, Form J can be obtained by precipitation of Form A from methyl ethyl ketone by the addition of toluene as an anti-solvent.

In one embodiment, Form J melts at about 130° C. to about 140° C. In another embodiment, Form J melts at about 134° C. to about 140° C. In another embodiment, Form J melts at about 134° C.

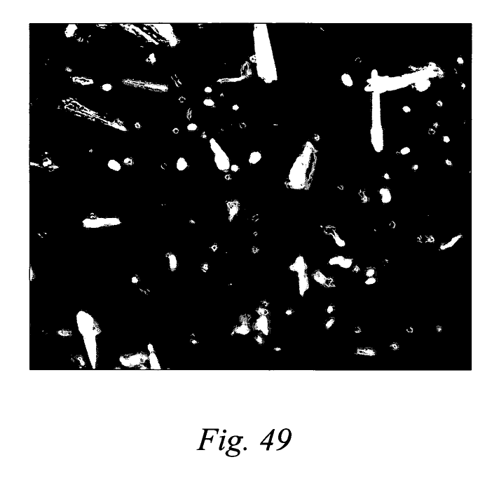

In another embodiment, Form J is a white, flaky crystalline solid with a particle size D₉₀<50 μm (see FIG. 49).

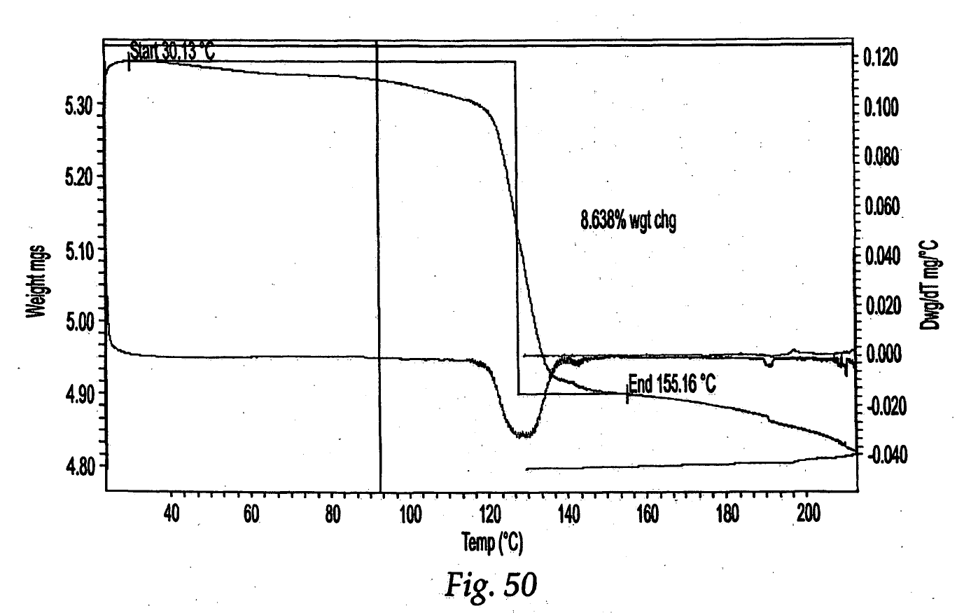

In another embodiment, Form J loses up to about 8.7% volatiles up to 155° C. by TGA (see FIG. 50).

In another embodiment, Form J is solvated.

In another embodiment, Form J is obtained in a molar ratio of 2.5:1 of Compound (I) and heptane as evidenced by TGA (see FIG. 50).

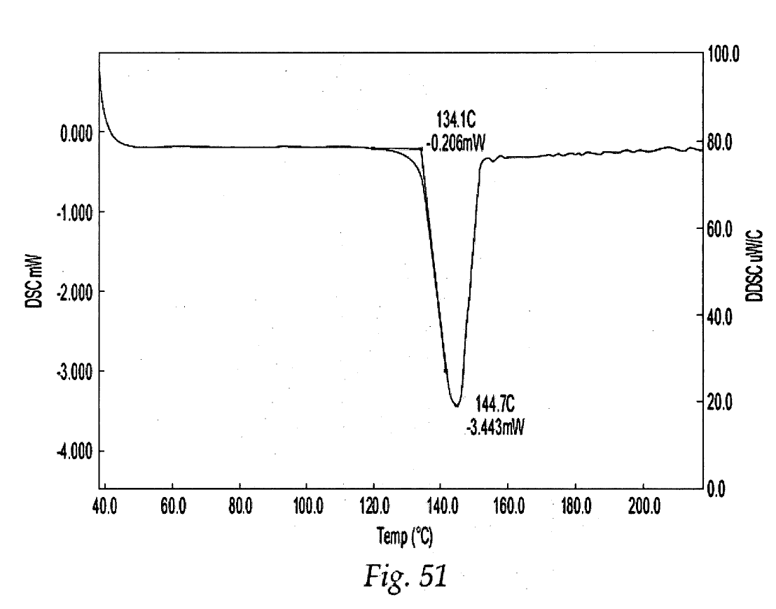

In another embodiment, Form J shows a single endothermal event at 134° C., and a melting temperature maximum of about 145° C. (see FIG. 51).

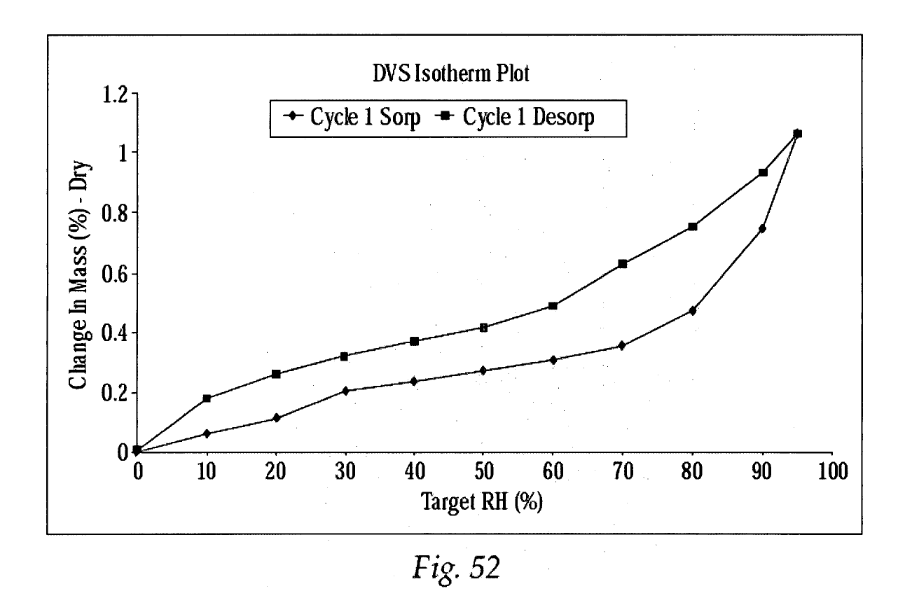

In another embodiment, Form J is slightly hygroscopic (i.e., exhibits an increase of about 1.1% in mass relative to the dry mass) at 25° C. up to 95% relative humidity (see FIG. 52).

In another embodiment, the XRPD diffractogram of Form J is unchanged after undergoing a full adsorption/desorption cycle.

In another embodiment, Form J can be converted to Form A by equilibrating in acetonitrile.

In certain embodiments, the present invention also contemplates obtaining one of Forms A-J of Compound (I) followed by conversion of that form to another form of Compound (I).

The exemplary methods and the examples described herein are illustrative of the present invention and are not to be construed as limiting the scope thereof.

5.2.3 Characterization Methods

The solid forms of the invention were characterized by XRPD on a Thermo ARL X'TRA X-ray powder diffractometer equipped with a fine focus X-ray tube using CuKα radiation at 1.54 Å. The voltage and amperage of the X-ray generator were set at 45 kV and 40 mA, respectively. The divergence slices were set at 4 mm and 2 mm and the measuring slices were set at 0.5 mm and 0.2 mm. The diffracted radiation was detected by a peltier-cooled Si(Li) solid state detector. Data was obtained using a theta-two theta continuous scan at 2.40°/min (0.5 sec/0.02° step) from 1.5° 2θ to 4° 2θ and a sintered alumina standard was used to check the peak position.

DSC analysis was performed on a Seiko Exstar DSC 6200R instrument using indium and tin as calibration standards. Approximately 1.5 to about 5 mg of sample was used for each experiment. Samples were heated under nitrogen at a rate of approximately 10° C./min up to a final temperature of about 200° C. Melting points are reported as the extrapolated onset temperature.

TG analysis was performed on a Thermo Cahn 2121 TGA instrument using calcium oxalate as a performance check. Approximately 4 to about 10 mg of sample was used for each experiment. Samples were heated under nitrogen at a rate of approximately 10° C./min up to a final temperature of about 200° C.

Morphology and particle size analyses of the samples were carried out on an Olympus microscope calibrated with USP standards.

Sample hygroscopicity was determined on a Surface Measurement System DVS. Approximately 10 to about 50 mg of sample was used for each experiment. Samples were analyzed on a DVS automated sorption analyzer at about 25° C. The relative humidity was increased in 10% increments from 0% to 95% relative humidity. The relative humidity was then decreased in a similar manner to accomplish a full adsorption/desorption cycle.

5.2.4 Pharmaceutical Compositions

In another aspect, the present invention provides pharmaceutical compositions comprising an effective amount of a solid form of the invention and a pharmaceutically acceptable carrier, diluent or excipient (referred to herein as a “pharmaceutical composition(s) of the invention”).

In one embodiment, the pharmaceutical compositions are useful for the treatment or prevention of a number of diseases, including, but not limited to, a liver disease, cancer, a cardiovascular disease, liver disease, cancer, cardiovascular diseases, metabolic diseases, renal diseases, autoimmune conditions, inflammatory conditions, fibrotic diseases, macular degeneration, pain and related syndromes, disease-related wasting, asbestos-related conditions, pulmonary hypertension, ischemia/reperfusion injury or central nervous system (CNS) injury/damage. The pharmaceutical compositions of the invention are also useful for inhibiting JNK and treating or preventing a disease associated with JNK, such as those treatable or preventable by the inhibition of JNK.

In certain embodiments, a pharmaceutical composition of the invention comprises a pure solid form of Compound (I). For example, a pharmaceutical composition of the invention can comprise pure Form A, pure Form B, pure Form C, pure Form D, pure Form E, pure Form F, pure Form G, pure Form H, pure Form I or pure Form J and a pharmaceutically acceptable carrier, diluent or excipient. In another embodiment, a pharmaceutical composition of the invention can comprise a mixture of two or more solid forms of the invention. For example, a pharmaceutical composition of the invention can comprise two or more of Form A, Form B, Form C, Form D, Form E, Form F, Form G, Form H, Form I or Form J and a pharmaceutically acceptable carrier, diluent or excipient.

Each solid form of the invention has an optimal therapeutic blood concentration and a lethal concentration. The bioavailability of the solid form of the invention determines the dosage strength in the pharmaceutical composition of the invention necessary to obtain the ideal blood level. If the pharmaceutical composition of the invention comprises two or more solid forms of the invention differing in bioavailability, the optimal dose will depend on the solid form present in the pharmaceutical composition of the invention.

The pharmaceutical compositions for the administration of the solid forms of the invention can be administered in a unit dosage form and can be prepared by any of the methods well known in the art of pharmacy. Methods can include the step of bringing a solid form of the invention into association with the carrier, diluent or excipient which constitutes one or more accessory ingredients. In general, the pharmaceutical compositions of the invention are prepared by uniformly and intimately bringing a solid form of the invention into association with a liquid carrier or a finely divided solid carrier or both, and then, if necessary, shaping the product into the desired formulation. In the pharmaceutical compositions of the invention, the solid form is present in an effective amount (i.e., an amount sufficient to treat or prevent the disease or disorder).

The pharmaceutical compositions of the invention containing a solid form of the invention can be in a form suitable for oral use, for example, as tablets, troches, lozenges, aqueous or oily suspensions, dispersible powders or granules, emulsions, hard or soft capsules, or syrups or elixirs. Pharmaceutical compositions intended for oral use can be prepared according to any method known to the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets can contain the a solid form of the invention in admixture with non-toxic pharmaceutically acceptable excipients which are suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they may be coated by known techniques to delay disintegration and absorption in the gastrointestinal tract and thereby provide a sustained action over a longer period. For example, a time delay material such as glyceryl monostearate or glyceryl distearate can be employed. They can also be coated by the techniques described in U.S. Pat. Nos. 4,256,108; 4,166,452 and 4,265,874 to form osmotic therapeutic tablets for control release.

Formulations for oral use can also be presented as hard gelatin capsules wherein the a solid form of the invention is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

In one embodiment, the invention provides single unit dosage forms comprising a solid form of the invention suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Aqueous suspensions can contain a solid form of the invention in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl, p-hydroxybenzoate, one or more coloring agents, one or more flavoring agents, and one or more sweetening agents, such as sucrose or saccharin.

Oily suspensions can be formulated by suspending a solid form of the invention in a vegetable oil, for example arachis oil, olive oil, sesame oil or coconut oil, or in a mineral oil such as liquid paraffin. The oily suspensions can contain a thickening agent, including, but not limited to, beeswax, hard paraffin or cetyl alcohol. Sweetening agents, such as those set forth above, and flavoring agents can be added to provide a palatable oral preparation. The pharmaceutical compositions of the invention can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, can also be present.

The pharmaceutical compositions of the invention can also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example, glycerol, propylene glycol, sorbitol or sucrose. Such formulations can also contain a demulcent, a preservative and flavoring and coloring agents.

The pharmaceutical compositions of the invention can be in the form of a sterile injectable aqueous or oleaginous suspension. This suspension can be formulated according to the known art using those suitable dispersing or wetting agents and suspending agents which have been mentioned above. The sterile injectable preparation can also be a sterile injectable solution or suspension in a non-toxic parenterally-acceptable diluent or solvent, for example as a solution in 1,3-butane diol. Among the acceptable diluents and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

The solid forms of the invention can also be administered in the form of suppositories for rectal administration of the drug. These compositions can be prepared by mixing the solid forms of the invention with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, containing a solid form of the invention are employed. As used herein, topical application is also meant to include the use of mouth washes and gargles.

The pharmaceutical compositions and methods of the present invention can further comprise one or more other therapeutically active compounds as noted herein which are usually applied in the treatment or prevention of the above mentioned diseases.

5.2.4 Methods of Use

In yet another aspect, the present invention provides methods for treating or preventing a liver disease (such as hepatitis, alcohol-induced liver disease, toxin-induced liver disease, steatosis or sclerosis); a cardiovascular disease (such as atherosclerosis, restenosis following angioplasty, left ventricular hypertrophy, myocardial infarction, chronic obstructive pulmonary disease, primary pulmonary hypertension or stroke); an angiogenic disease; ischemic damage (such as to the heart, lung, gut, kidney, liver, pancreas, spleen or brain); ischemia-reperfusion injury (such as that caused by transplant, surgical trauma, hypotension, thrombosis or trauma injury); a neurodegenerative disease (such as epilepsy, Alzheimer's disease, Huntington's disease, Amyotrophic lateral sclerosis, peripheral neuropathies, spinal cord damage, AIDS dementia complex or Parkinson's disease); an inflammatory disease (such as Type II diabetes, Type I diabetes, diabetes insipidus, diabetes mellitus, maturity-onset diabetes, juvenile diabetes, insulin-dependant diabetes, insulin resistance, non-insulin dependant diabetes, malnutrition-related diabetes, ketosis-prone diabetes, ketosis-resistant diabetes, nephropathy, nephritis, glomerulonephritis, graft versus host disease, acute/chronic kidney failure, obesity, hereditary obesity, dietary obesity, hormone related obesity, obesity related to the administration of medication, hearing loss, otitis externa, acute otitis media, wound-healing, burn-healing (e.g., wherein the burn is a first-, second- or third-degree burn and/or a thermal, chemical or electrical burn), arthritis, rheumatoid arthritis, rheumatoid spondylitis, osteoarthritis, gout, allergy; allergic rhinitis, acute respiratory distress syndrome, asthma, bronchitis, inflammatory bowel disease, irritable bowel syndrome, mucous colitis, ulcerative colitis, Crohn's disease, gastritis, esophagitis, pancreatitis, peritonitis); a fibrotic disease (such as idiopathic pulmonary fibrosis, pulmonary interstitial fibrosis, renal fibrosis, cystic fibrosis, liver fibrosis or a fibrotic disease of the liver, skin, lung, kidney, heart, pancreas, bone marrow or peritoneum) an autoimmune disease (such as scleroderma, systemic lupus erythematosus, myasthenia gravis, Grave's disease, transplant rejection, endotoxin shock, sepsis, psoriasis, eczema, dermatitis or multiple sclerosis); disease-related wasting (HIV or AIDS related wasting); cachexia; myeloproliferative disorders; myelodysplastic syndromes; complex regional pain syndrome (including symptoms associated with complex regional pain syndrome, such as but are not limited to, pain, autonomic dysfunction, trigeminal neuralgia, post-herpetic neuralgia, cancer-related pain, phantom limb pain, fibromyalgia, chronic fatigue syndrome, radiculopathy, inability to initiate movement, weakness, tremor, muscle spasm, dytonia, dystrophy, atrophy, edema, stiffness, joint tenderness, increased sweating, sensitivity to temperature, light touch, color change to the skin, hyperthermic or hypothermic, increased nail or hair growth, early bony changes, hyperhidrotic with livedo reticularis or cyanosis, lost hair, ridged, cracked or brittle nails, dry hand, diffuse osteoporosis, irreversible tissue damage, thin and shiny skin, joint contractures, marked demineralization and other painful neuropathic conditions such as diabetic neuropathy); macular degeneration; cancer (e.g., cancer of the head, neck, throat, larynx, eye, mouth, throat, esophagus, pharynx, chest, bone, lung, bronchus, colon, rectum, stomach, prostate, breast, ovaries, cervix, uterine, testicles or other reproductive organs, skin, thyroid, blood, lymph nodes, kidney, liver, pancreas, urinary bladder and brain or central nervous system); a myeloproliferative disorder (e.g., polycythemia rubra vera, primary thrombocythemia, chronic myelogenous leukemia, acute myelogenous leukemia, acute or chronic granulocytic leukemia, acute or chronic myelomonocytic leukemia, myelofibro-erythroleukemia, or agnogenic myeloid metaplasia); or a myelodysplastic syndrome (e.g., refractory anemia, refractory anemia with ringed sideroblasts, refractory anemia with excess blasts, refractory anemia with excess blasts in transformation, preleukemia or chronic myelomonocytic leukemia), comprising administering an effective amount of a solid form of the invention to a patient in need thereof.

In another embodiment, the present invention provides methods for inhibiting JNK in a cell (e.g., a mammalian cell), comprising contacting the cell with an effective amount of a solid form of the invention.

Cancers within the scope of the invention include those associated with BCR-ABL, and mutants or isoforms thereof, as well as kinases from the src kinase family, kinases from the Rsk kinase family, kinases from the CDK family, kinases from the MAPK kinase family, and tyrosine kinases such as Fes, Lyn, and Syk kinases, and mutants or isoforms thereof.

In a particular embodiment, the invention relates to the treatment or prevention of a disease or disorder associated with the modulation, for example inhibition, of a kinase, including, but are not limited to, tyrosine-protein kinase (SYK), tyrosine-protein kinase (ZAP-70), protein tyrosine kinase 2 beta (PYK2), focal adhesion kinase 1 (FAK), B lymphocyte kinase (BLK), hemopoietic cell kinase (HCK), v-yes-1 Yamaguchi sarcoma viral related oncogene homolog (LYN), T cell-specific protein-tyrosine kinase (LCK), proto-oncogene tyrosine-protein kinase (YES), proto-oncogene tyrosine-protein kinase (SRC), proto-oncogene tyrosine-protein kinase (FYN), proto-oncogene tyrosine-protein kinase (FGR), proto-oncogene tyrosine-protein kinase (FER), proto-oncogene tyrosine-protein kinase (FES), C-SRC kinase, protein-tyrosine kinase (CYL), tyrosine protein kinase (CSK), megakaryocyte-associated tyrosine-protein kinase (CTK), tyrosine-protein kinase receptor (EPH), Ephrin type-A receptor 1, Ephrin type-A receptor 4 (EPHA4), Ephrin type-B receptor 3 (EPHB3), Ephrin type-A receptor 8 (EPHA8), neurotrophic tyrosine kinase receptor, type 1 (NTRK1), protein-tyrosine kinase (PTK2), syk-related tyrosine kinase (SRK), protein tyrosine kinase (CTK), tyro3 protein tyrosine kinase (TYRO3), bruton agammaglobulinemia tyrosine kinase (BTK), leukocyte tyrosine kinase (LTK), protein-tyrosine kinase (SYK), protein-tyrosine kinase (STY), tek tyrosine kinase (TEK), elk-related tyrosine kinase (ERK), tyrosine kinase with immunoglobulin and egf factor homology domains (TIE), protein tyrosine kinase (TKF), neurotrophic tyrosine kinase, receptor, type 3 (NTRK3), mixed-lineage protein kinase-3 (MLK3), protein kinase, mitogen-activated 4 (PRKM4), protein kinase, mitogen-activated 1 (PRKM1), protein tyrosine kinase (PTK7), protein tyrosine kinase (EEK), minibrain (drosophila) homolog (MNBH), bone marrow kinase, x-linked (BMX), eph-like tyrosine kinase 1 (ETK1), macrophage stimulating 1 receptor (MST1R), btk-associated protein, 135 kd, lymphocyte-specific protein tyrosine kinase (LCK), fibroblast growth factor receptor-2 (FGFR2), protein tyrosine kinase-3 (TYK3), protein tyrosine kinase (TXK), tec protein tyrosine kinase (TEC), protein tyrosine kinase-2 (TYK2), eph-related receptor tyrosine kinase ligand 1 (EPLG1), t-cell tyrosine kinase (EMT), eph tyrosine kinase 1 (EPHT1), zona pellucida receptor tyrosine kinase, 95 kd (ZRK), protein kinase, mitogen-activated, kinase 1 (PRKMK1), eph tyrosine kinase 3 (EPHT3), growth arrest-specific gene-6 (GAS6), kinase insert domain receptor (KDR), axl receptor tyrosine kinase (AXL), fibroblast growth factor receptor-1 (FGFR1), v-erb-b2 avian erythroblastic leukemia viral oncogene homolog 2 (ERBB2), fins-like tyrosine kinase-3 (FLT3), neuroepithelial tyrosine kinase (NEP), neurotrophic tyrosine kinase receptor-related 3 (NTRKR3), eph-related receptor tyrosine kinase ligand 5 (EPLG5), neurotrophic tyrosine kinase, receptor, type 2 (NTRK2), receptor-like tyrosine kinase (RYK), tyrosine kinase, b-lymphocyte specific (BLK), eph tyrosine kinase 2 (EPHT2), eph-related receptor tyrosine kinase ligand 2 (EPLG2), glycogen storage disease VIII, eph-related receptor tyrosine kinase ligand 7 (EPLG7), janus kinase 1 (JAK1), fins-related tyrosine kinase-1 (FLT1), protein kinase, camp-dependent, regulatory, type I, alpha (PRKAR1A), wee-1 tyrosine kinase (WEE1), eph-like tyrosine kinase 2 (ETK2), receptor tyrosine kinase musk, insulin receptor (INSR), janus kinase 3 (JAK3), fms-related tyrosine kinase-3 ligand protein kinase c, beta 1 (PRKCB1), tyrosine kinase-type cell surface receptor (HER3), janus kinase 2 (JAK2), lim domain kinase 1 (LIMK1), dual specificity phosphatase 1 (DUSP1), hemopoietic cell kinase (HCK), tyrosine 3-monooxygenase/tryptophan 5-monooxygenase activation protein, eta polypeptide (YWHAH), ret proto-oncogene (RET), tyrosine 3-monooxygenase/tryptophan 5-monooxygenase activation protein, zeta polypeptide (YWHAZ), tyrosine 3-monooxygenase/tryptophan 5-monooxygenase activation protein, beta polypeptide (YWHAB), hepatoma transmembrane kinase (HTK), map kinase kinase 6, phosphatidylinositol 3-kinase, catalytic, alpha polypeptide (PIK3CA), cyclin-dependent kinase inhibitor 3 (CDKN3), diacylglycerol kinase, delta, 130 kd, protein-tyrosine phosphatase, nonreceptor type, 13 (PTPN13), abelson murine leukemia viral oncogene homolog 1 (ABL1), diacylglycerol kinase, alpha (DAGK1), focal adhesion kinase 2, epithelial discoidin domain receptor 1 (EDDR1), anaplastic lymphoma kinase (ALK), phosphatidylinositol 3-kinase, catalytic, gamma polypeptide (PIK3CG), phosphatidylinositol 3-kinase regulatory subunit, (PIK3R1), eph homology kinase-1 (EHK1), v-kit hardy-zuckerman 4 feline sarcoma viral oncogene homolog (KIT), fibroblast growth factor receptor-3 (FGFR3), vascular endothelial growth factor c (VEGFC), epidermal growth factor receptor (EGFR), oncogene (TRK), growth factor receptor-bound protein-7 (GRB7), ras p21 protein activator (RASA2), met proto-oncogene (MET), src-like adapter (SLA), vascular endothelial growth factor (VEGF), vascular endothelial growth factor receptor (VEGFR), nerve growth factor receptor (NGFR), platelet derived growth factor receptor (PDGFR), platelet derived growth factor receptor beta (PDGFRB), dual-specificity tyrosine-(Y)-phosphorylation regulated kinase 2 (DYRK2), dual-specificity tyrosine-(Y)-phosphorylation regulated kinase 3 (DYRK3), dual-specificity tyrosine-(Y)-phosphorylation regulated kinase 4 (DYRK4), dual-specificity tyrosine-(Y)-phosphorylation regulated kinase 1A (DYRK1A), dual-specificity tyrosine-(Y)-phosphorylation regulated kinase 1B (DYRK1B), CDC-like kinase 1 (CLK1), protein tyrosine kinase STY, CDC-like kinase 4 (CLK4), CDC-like kinase 2 (CLK2) or CDC-like kinase 3 (CLK3).

In another embodiment, the invention relates to the treatment or prevention of a disease or disorder associated with the modulation, for example inhibition, of serine/threonine kinases or related molecules, including, but not limited to, cyclin-dependent kinase 7 (CDK7), rac serine/threonine protein kinase, serine-threonine protein kinase n (PKN), serine/threonine protein kinase 2 (STK2), zipper protein kinase (ZPK), protein-tyrosine kinase (STY), bruton agammaglobulinemia tyrosine kinase (BTK), mkn28 kinase, protein kinase, x-linked (PRKX), elk-related tyrosine kinase (ERK), ribosomal protein s6 kinase, 90 kd, polypeptide 3 (RPS6KA3), glycogen storage disease VIII, death-associated protein kinase 1 (DAPK1), pctaire protein kinase 1 (PCTK1), protein kinase, interferon-inducible double-stranded rna (PRKR), activin a receptor, type II-like kinase 1 (ACVRLK1), protein kinase, camp-dependent, catalytic, alpha (PRKACA), protein kinase, y-linked (PRKY), G protein-coupled receptor kinase 2 (GPRK21), protein kinase c, theta form (PRKCQ), lim domain kinase 1 (LIMK1), phosphoglycerate kinase 1 PGK1), lim domain kinase 2 (LIMK2), c-jun kinase, activin a receptor, type II-like kinase 2 (ACVRLK2), janus kinase 1 (JAK1), elk1 motif kinase (EMK1), male germ cell-associated kinase (MAK), casein kinase 2, alpha-prime subunit (CSNK2A2), casein kinase 2, beta polypeptide (CSNK2B), casein kinase 2, alpha 1 polypeptide (CSNK2A1), ret proto-oncogene (RET), hematopoietic progenitor kinase 1, conserved helix-loop-helix ubiquitous kinase (CHUK), casein kinase 1, delta (CSNK1D), casein kinase 1, epsilon (CSNK1E), v-akt murine thymoma viral oncogene homolog 1 (AKT1), tumor protein p53 (TP53), protein phosphatase 1, regulatory (inhibitor) subunit 2 (PPP1R2), oncogene pim-1 (PIM1), transforming growth factor-beta receptor, type II (TGFBR2), transforming growth factor-beta receptor, type I (TGFBR1), v-raf murine sarcoma viral oncogene homolog b1 (BRAF), bone morphogenetic receptor type II (BMPR2), v-raf murine sarcoma 3611 viral oncogene homolog 1 (ARAF1), v-raf murine sarcoma 3611 viral oncogene homolog 2 (ARAF2), protein kinase C (PKC), v-kit hardy-zuckerman 4 feline sarcoma viral oncogene homolog (KIT) or c-KIT receptor (KITR).

In another embodiment, the invention relates to the treatment or prevention of a disease or disorder associated with the modulation, for example inhibition, of a MAP kinase, including, but not limited to, mitogen-activated protein kinase 3 (MAPK3), p44erk1, p44mapk, mitogen-activated protein kinase 3 (MAP kinase 3; p44), ERK1, PRKM3, P44ERK1, P44MAPK, mitogen-activated protein kinase 1 (MAPK1), mitogen-activated protein kinase kinase 1 (MEK1), MAP2K1 protein tyrosine kinase ERK2, mitogen-activated protein kinase 2, extracellular signal-regulated kinase 2, protein tyrosine kinase ERK2, mitogen-activated protein kinase 2, extracellular signal-regulated kinase 2, ERK, p38, p40, p41, ERK2, ERT1, MAPK2, PRKM1, PRKM2, P42MAPK, p41mapk, mitogen-activated protein kinase 7 (MAPK7), BMK1 kinase, extracellular-signal-regulated kinase 5, BMK1, ERK4, ERK5, PRKM7, nemo-like kinase (NLK), likely ortholog of mouse nemo like kinase, mitogen-activated protein kinase 8 (MAPK8), protein kinase JNK1, JNK1 beta protein kinase, JNK1 alpha protein kinase, c-Jun N-terminal kinase 1, stress-activated protein kinase JNK1, JNK, JNK1, PRKM8, SAPK1, JNK1A2, JNK21B1/2, mitogen-activated protein kinase 10 (MAPK10), c-Jun kinase 3, JNK3 alpha protein kinase, c-Jun N-terminal kinase 3, stress activated protein kinase JNK3, stress activated protein kinase beta, mitogen-activated protein kinase 9 (MAPK9), MAP kinase 9, c-Jun kinase 2, c-Jun N-terminal kinase 2, stress-activated protein kinase JNK2, JNK2, JNK2A, JNK2B, PRKM9, JNK-55, JNK2BETA, p54aSAPK, JNK2ALPHA, mitogen-activated protein kinase 14 (MAPK14), p38 MAP kinase, MAP kinase Mxi2, Csaids binding protein, MAX-interacting protein 2, stress-activated protein kinase 2A, p38 mitogen activated protein kinase, cytokine suppressive anti-inflammatory drug binding protein, RK, p38, EXIP, Mxi2, CSBP1, CSBP2, CSPB1, PRKM14, PRKM15, SAPK2A, p38ALPHA, mitogen-activated protein kinase 11 (MAPK11), stress-activated protein kinase-2, stress-activated protein kinase-2b, mitogen-activated protein kinase p38-2, mitogen-activated protein kinase p38beta, P38B, SAPK2, p38-2, PRKM11, SAPK2B, p38Beta, P38BETA2, mitogen-activated protein kinase 13 (MAPK 13), stress-activated protein kinase 4, mitogen-activated protein kinase p38 delta, SAPK4, PRKM13, p38delta, mitogen-activated protein kinase 12 (MAPK12), p38gamma, stress-activated protein kinase 3, mitogen-activated protein kinase 3, ERK3, ERK6, SAPK3, PRKM12, SAPK-3, P38GAMMA, mitogen-activated protein kinase 6 (MAPK6), MAP kinase isoform p97, mitogen-activated 5 protein kinase, mitogen-activated 6 protein kinase, extracellular signal-regulated kinase 3, extracellular signal-regulated kinase, p97, ERK3, PRKM6, p97MAPK, mitogen-activated protein kinase 4 (MAPK4), Erk3-related protein kinase, mitogen-activated 4 protein kinase (MAP kinase 4; p63), PRKM4, p63MAPK, ERK3-RELATED or Extracellular signal-regulated kinase 8 (ERK7).

More particularly, cancers and related disorders that can be treated or prevented by methods and compositions of the present invention include, but are not limited to, the following: Leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome (or a symptom thereof such as anemia, thrombocytopenia, neutropenia, bicytopenia or pancytopenia), refractory anemia (RA), RA with ringed sideroblasts (RARS), RA with excess blasts (RAEB), RAEB in transformation (RAEB-T), preleukemia and chronic myelomonocytic leukemia (CMML), chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer, including, but not limited to, adenocarcinoma, lobular (small cell) carcinoma, intraductal carcinoma, medullary breast cancer, mucinous breast cancer, tubular breast cancer, papillary breast cancer, primary cancers, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Accordingly, the methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal orignin, including fibrosarcoma and rhabdomyoscarcoma; other tumors, including melanoma, seminoma, tetratocarcinoma, neuroblastoma and glioma; tumors of the central and peripheral nervous system, including astrocytoma, glioblastoma multiforme, neuroblastoma, glioma, and schwannomas; solid and blood born tumors; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the ovary, bladder, breast, colon, lung, skin, pancreas, or uterus. In other specific embodiments, sarcoma, melanoma, or leukemia is treated or prevented.

In another embodiment, the methods and compositions of the invention are also useful for administration to patients in need of a bone marrow transplant to treat a malignant disease (e.g., patients suffering from acute lymphocytic leukemia, acute myelogenous leukemia, chronic myelogenous leukemia, chronic lymphocytic leukemia, myelodysplastic syndrome (“preleukemia”), monosomy 7 syndrome, non-Hodgkin's lymphoma, neuroblastoma, brain tumors, multiple myeloma, testicular germ cell tumors, breast cancer, lung cancer, ovarian cancer, melanoma, glioma, sarcoma or other solid tumors), those in need of a bone marrow transplant to treat a non-malignant disease (e.g., patients suffering from hematologic disorders, congenital immunodeficiences, mucopolysaccharidoses, lipidoses, osteoporosis, Langerhan's cell histiocytosis, Lesch-Nyhan syndrome or glycogen storage diseases), those undergoing chemotherapy or radiation therapy, those preparing to undergo chemotherapy or radiation therapy and those who have previously undergone chemotherapy or radiation therapy.

In another embodiment, the invention also provides methods for the treatment of myeloproliferative disorders or myelodysplastic syndromes, comprising administering to a patient in need thereof an effective amount of a solid form of the invention or a composition thereof. In certain embodiments, the myeloproliferative disorder is polycythemia rubra vera; primary thrombocythemia; chronic myelogenous leukemia; acute or chronic granulocytic leukemia; acute or chronic myelomonocytic leukemia; myelofibro-erythroleukemia; or agnogenic myeloid metaplasia.

In another embodiment, the invention also provides methods for the treatment of cancer or tumors resistant to other kinase inhibitors such as imatinib mesylate (STI-571 or Gleevec™) treatment, comprising administering to a patient in need thereof an effective amount of a solid form of the invention or a composition thereof. In a particular embodiment, the invention provides methods for the treatment of leukemias, including, but not limited to, gastrointestinal stromal tumor (GIST), acute lymphocytic leukemia or chronic myelocytic leukemia resistant to imatinib mesylate (STI-571 or Gleevec™) treatment, comprising administering to a patient in need thereof an effective amount of a solid form of the invention or a composition thereof.

In one embodiment, the invention relates to methods for treating or preventing a disease or disorder treatable or preventable by modulating a kinase pathway, in one embodiment, the JNK pathway, comprising administering an effective amount of a solid form of the invention or a composition thereof to a patient in need of the treating or preventing. Particular diseases which are treatable or preventable by modulating, for example, inhibiting, a kinase pathway, in one embodiment, the JNK pathway, include, but are not limited to, rheumatoid arthritis; rheumatoid spondylitis; osteoarthritis; gout; asthma, bronchitis; allergic rhinitis; chronic obstructive pulmonary disease; cystic fibrosis; inflammatory bowel disease; irritable bowel syndrome; mucous colitis; ulcerative colitis; Crohn's disease; Huntington's disease; gastritis; esophagitis; hepatitis; pancreatitis; nephritis; multiple sclerosis; lupus erythematosus; Type II diabetes; obesity; atherosclerosis; restenosis following angioplasty; left ventricular hypertrophy; myocardial infarction; stroke; ischemic damages of heart, lung, gut, kidney, liver, pancreas, spleen and brain; acute or chronic organ transplant rejection; preservation of the organ for transplantation; organ failure or loss of limb (e.g., including, but not limited to, that resulting from ischemia-reperfusion injury, trauma, gross bodily injury, car accident, crush injury or transplant failure); graft versus host disease; endotoxin shock; multiple organ failure; psoriasis; burn from exposure to fire, chemicals or radiation; eczema; dermatitis; skin graft; ischemia; ischemic conditions associated with surgery or traumatic injury (e.g., vehicle accident, gunshot wound or limb crush); epilepsy; Alzheimer's disease; Parkinson's disease; immunological response to bacterial or viral infection; cachexia; angiogenic and proliferative dieseases; solid tumor; and cancers of a variety of tissues such as colon, rectum, prostate, liver, lung, bronchus, pancreas, brain, head, neck, stomach, skin, kidney, cervix, blood, larynx, esophagus, mouth, pharynx, urinary bladder, ovary or uterine.

Depending on the disease to be treated and the patient's condition, the solid forms of the present invention may be administered by oral, parenteral (e.g., intramuscular, intraperitoneal, intravenous, ICV, intracisternal injection or infusion, subcutaneous injection, or implant), inhalation spray, nasal, vaginal, rectal, sublingual, or topical routes of administration and can be formulated, alone or together, in suitable dosage unit formulations containing conventional non-toxic pharmaceutically acceptable carriers, adjuvants and vehicles appropriate for each route of administration.

In the treatment or prevention of diseases which require the inhibition of JNK, an appropriate dosage level will generally be about 0.001 to 100 mg/kg patient body weight per day, which can be administered in single or multiple doses. In another embodiment, the dosage level will be about 0.01 to about 25 mg/kg per day; in a further embodiment about 0.05 to about 10 mg/kg per day. A suitable dosage level can be about 0.01 to 25 mg/kg per day, about 0.05 to 10 mg/kg per day, or about 0.1 to 5 mg/kg per day. Within this range the dosage can be 0.005 to 0.05, 0.05 to 0.5 or 0.5 to 5.0 mg/kg per day. For oral administration, the compositions are preferably provided in the form of tablets containing about 1.0 to about 1000 milligrams of the active ingredient, particularly about 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the solid form of the invention for the symptomatic adjustment of the dosage to the patient to be treated. The solid forms of the invention can be administered on a regimen of 1 to 4 times per day, in one embodiment once or twice per day.

It will be understood, however, that the specific dose level and frequency of dosage for any particular patient can be varied and will depend upon a variety of factors including the activity of the specific solid form employed, the metabolic stability and length of action of that solid form, the age, body weight, general health, sex, diet, mode and time of administration, rate of excretion, drug combination, the severity of the particular disease, and the host undergoing treatment.

The solid forms of the invention can be combined with other compounds having related utilities to treat or prevent inflammatory and immune disorders and diseases, including asthma and allergic diseases and immunological responses to bacterial or viral infection, as well as autoimmune pathologies such as rheumatoid arthritis and atherosclerosis, and those pathologies noted above. In many instances, compositions which include a solid form of the invention and an alternative or second therapeutic agent have additive or synergistic effects when administered.

For example, in the treatment or prevention of inflammation, the present solid forms can be used in conjunction or combination with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codiene, fentanyl, ibuprofen, indomethacin, ketorolac, morphine, naproxen, phenacetin, piroxicam, a steroidal analgesic, sufentanyl, sunlindac, tenidap, and the like. Similarly, the instant solid forms of the invention can be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextromethorphan; a diuretic; and a sedating or non-sedating antihistamine. Likewise, solid forms of the invention can be used in combination with other drugs that are used in the treatment/prevention/suppression or amelioration of the diseases or conditions for which solid forms of the present invention are useful. Such other drugs can be administered, by a route and in an amount commonly used therefore, contemporaneously or sequentially with a solid form of the present invention. When a solid form of the invention is used contemporaneously with one or more other drugs, a pharmaceutical composition containing such other drugs in addition to the solid form of the invention is preferred. Accordingly, the pharmaceutical compositions of the present invention include those that also contain one or more other active ingredients, in addition to a solid form of the invention. Examples of other active ingredients that can be combined with a solid form of the invention, either administered separately or in the same pharmaceutical compositions, include, but are not limited to: (a) VLA-4 antagonists, (b) steroids such as beclomethasone, methylprednisolone, betamethasone, prednisone, dexamethasone, and hydrocortisone; (c) immunosuppressants such as cyclosporine (cyclosporine A, Sandimmune®, Neoral®, tacrolimus (FK-506, Prograf®), rapamycin (sirolimus, Rapamune®) and other FK-506 type immunosuppressants, and mycophenolate, e.g., mycophenolate mofetil (CellCept®); (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as β2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors such as celecoxib (Celebrex®) and rofecoxib (Vioxx®); (h) inhibitors of phosphodiesterase type IV (PDE-IV); (i) gold compounds such as auranofin and aurothioglucose, 0) inhibitors of phosphodiesterase type IV (PDE-IV); (k) other antagonists of the chemokine receptors, especially CCR1, CCR2, CCR3, CCR5, CCR6, CCR8 and CCR10; (1) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (m) anti-diabetic agents such as insulin, sulfonylureas, biguamides (metformin), α-glucosidase inhibitors (acarbose) and glitazones (troglitazone and pioglitazone); (n) preparations of interferon beta (interferon β-1α, interferon β-1β); (O) etanercept (Enbrel®), (p) antibody therapies such as orthoclone (OKT3), daclizumab (Zenapax®), infliximab (Remicade®), basiliximab (Simulect®) and anti-CD40 ligand antibodies (e.g., MRP-1); and (q) other compounds such as 5-aminosalicylic acid and prodrugs thereof, hydroxychloroquine, D-penicillamine, antimetabolites such as azathioprine and 6-mercaptopurine, cytotoxic cancer chemotherapeutic agents, bosentan, INF-gamma, imatinib, anti-CTGF (FG-3019), anti-TGFβ and pirfenidone. The weight ratio of the solid form of the present invention to the second active ingredient may be varied and will depend upon the effective dose of each ingredient. Generally, an effective dose of each will be used. Thus, for example, when a solid form of the present invention is combined with an NSAID the weight ratio of the solid form of the invention to the NSAID will generally range from about 1000:1 to about 1:1000, preferably about 200:1 to about 1:200. Combinations of a solid form of the invention and other active ingredients will generally also be within the aforementioned range, but in each case, an effective dose of each active ingredient should be used.

Immunosuppressants within the scope of the present invention further include, but are not limited to, leflunomide, RAD001, ERL080, FTY720, CTLA-4, antibody therapies such as orthoclone (OKT3), daclizumab (Zenapax®) and basiliximab (Simulect®), and antithymocyte globulins such as thymoglobulins.

In certain embodiments, the present methods are directed to the treatment or prevention of multiple sclerosis using a solid form of the invention either alone or in combination with a second therapeutic agent selected from betaseron, avonex, azathioprine (Imurek®, Imuran®), capoxone, prednisolone and cyclophosphamide. When used in combination, the practitioner can administer a combination of the therapeutic agents, or administration can be sequential.

In another embodiment, the present methods are directed to the treatment or prevention of rheumatoid arthritis, wherein the solid form of the invention is administered either alone or in combination with a second therapeutic agent selected from the group consisting of methotrexate, sulfasalazine, hydroxychloroquine, cyclosporine A, D-penicillamine, infliximab (Remicade(®), etanercept (Enbrel®), auranofin and aurothioglucose.

In another embodiment, the present methods are directed to the treatment or prevention of an organ transplant condition wherein the solid form of the invention is used alone or in combination with a second therapeutic agent selected from the group consisting of cyclosporine A, FK-506, rapamycin, mycophenolate, prednisolone, azathioprine, cyclophosphamide and an antilymphocyte globulin.

In another embodiment, the invention relates to methods for preserving tissue, comprising contacting ex vivo tissue with an effective amount of a solid form of the invention.

In another embodiment, the invention relates to methods for preventing reperfusion injury to implanted tissue, comprising: (a) contacting tissue with an effective amount of a solid form of the invention; and (b) implanting the contacted tissue in a recipient.

In another embodiment, the invention relates to methods for preventing transplant rejection, comprising: (a) administering to a transplant recipient in need thereof an effective amount of a solid form of the invention; and (b) transplanting tissue in a recipient.

In another embodiment, the invention relates to methods for preserving tissue, comprising: (a) administering an effective amount of a solid form of the invention to a tissue donor; and (b) removing the tissue from the donor.

In another embodiment, the invention relates to a composition comprising ex vivo tissue and an effective amount of a solid form of the invention.

In another embodiment, the invention relates to a method for treating or preventing organ failure comprising administering an effective amount of a solid form of the invention to a patient in need thereof.

In another embodiment, the invention relates to a method for preventing ischemia-reperfusion injury that occurs during or as a result of surgery or trauma from accident comprising administering an effective amount of a solid form of the invention to a patient in need thereof.

In another embodiment, the invention relates to a container containing ex vivo tissue and an effective amount of a solid form of the invention.

In another embodiment, the invention relates to methods for preserving a cell to be implanted, comprising: (a) contacting a cell with an effective amount of a solid form of the invention; and (b) implanting the contacted cell in a recipient.

In another embodiment, the invention relates to methods for preserving an organ to be implanted, comprising: (a) contacting an organ with an effective amount of a solid form of the invention; and (b) implanting the contacted organ in a recipient.

In another embodiment, the invention relates to a stent or stent graft coated with an effective amount of a solid form of the invention. In a particular embodiment, the stent or stent graft is optionally further coated with an effective amount of an anticoagulant agent, an antimetabolite agent, an anti-inflammatory agent, an antiplatelet agent, an antithrombin agent, an antimitotic agent, a cytostatic agent or an antiproliferative agent.

### 6. EXAMPLES

Reagents and solvents used below can be obtained from commercial sources such as Aldrich Chemical Co. (Milwaukee, Wis., USA).

The solid forms of the invention were characterized by XRPD on a Thermo ARL X'TRA X-ray powder diffractometer equipped with a fine focus X-ray tube using CuKα radiation at 1.54 Å. The voltage and amperage of the X-ray generator were set at 45 kV and 40 mA, respectively. The divergence slices were set at 4 mm and 2 mm and the measuring slices were set at 0.5 mm and 0.2 mm. The diffracted radiation was detected by a peltier-cooled Si(Li) solid state detector. Data was obtained using a theta-two theta continuous scan at 2.40°/min (0.5 sec/0.02° step) from 1.5° 2θ to 4° 2θ and a sintered alumina standard was used to check the peak position.

DSC analyses were performed on a Seiko Exstar DSC 6200R instrument using indium and tin as calibration standards. Approximately 1.5 to about 5 mg of sample was used for each experiment. Samples were heated under nitrogen at a rate of approximately 10° C./min up to a final temperature of about 200° C. Melting points are reported as the extrapolated onset temperature.

TG analyses were performed on a Thermo Cahn 2121 TGA instrument using calcium oxalate as a performance check. Approximately 4 to about 10 mg of sample was used for each experiment. Samples were heated under nitrogen at a rate of approximately 10° C./min up to a final temperature of about 200° C.

Morphology and particle size analyses of the samples were carried out on an Olympus microscope calibrated with USP standards.

Sample hygroscopicity was determined on a Surface Measurement System DVS. Approximately 10 to about 50 mg of sample was used for each experiment. Samples were analyzed on a DVS automated sorption analyzer at about 25° C. The relative humidity was increased in 10% increments from 0% to 95% relative humidity. The relative humidity was then decreased in a similar manner to accomplish a full adsorption/desorption cycle.

### 6.1 Example 1

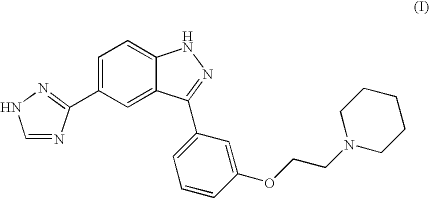

### A. 3-{1-Perhydro-2H-pyran-2-yl-5-[1-(triphenylmethyl)(1,2,4-triazol-3-yl)]-1H-indazol-3-yl}phenol

To a stirred solution of 2-{3-bromo-5-[1-(triphenylmethyl)(1,2,4-triazol-3-yl)]-1H-indazoyl}perhydro-2H-pyran (3.22 g, 5.46 mmol) in dimethoxyethane (27.1 mL) was added 3-hydroxy phenylboronic acid (1.81 g, 8.22 mmol), dichloro[1,1′-bis(diphenylphosphino)ferrocene]palladium (0.447 g, 0.485 mmol), and potassium phosphate (5.78 g, 27.2 mmol) and the mixture was heated at reflux for about 48 h. The mixture was diluted with dichloromethane. The organic extracts were washed with saturated sodium bicarbonate, dried over anhydrous sodium sulfate, filtered and evaporated. Purification of the residue using column chromatography with 20-50% ethyl acetate/hexanes provided the product (3.16 g, 96%, yield). ES-MS (m/z) 362 [M+1(-Tr)]⁺.

### B. 1-(5-(1H-1,2,4-Triazol-5-yl)(1H-indazol-3-yl))-3-(2-piperidylethoxy)benzene

Triphenylphosphine (0.694 g, 2.65 mmol), tetrahydrofuran (2.12 mL), 2-piperidylethanol (0.352 mL, 2.65 mmol) and diethylazodicarboxylate (0.418 mL, 2.65 mmol) were added to 3-{1-perhydro-2H-pyran-2-yl-5-[1-(triphenylmethyl)(1,2,4-triazol-3-yl)]-1H-indazol-3-yl}phenol (0.400 g, 0.662 mmol). The mixture was stirred at ambient temperature for about 23 h and poured into aqueous 6 N hydrochloric acid (30 mL). After stirring at ambient temperature for about 4 h, the mixture was extracted with ether (3×). The aqueous fraction was added to aqueous 6 N sodium hydroxide (30 mL) and the pH adjusted to 11. The solution was extracted with ethyl acetate (3×) and the organic fractions were combined and dried over anhydrous sodium sulfate, filtered and evaporated. The residue was purified using flash chromatography on silica pretreated with 2% triethylamine/ethyl acetate elution followed by 0-20% methanol/ethyl acetate. The desired fractions were concentrated, dissolved in ethyl acetate, washed with aqueous sodium bicarbonate, dried over anhydrous sodium sulfate, filtered and evaporated to provide the title compound (Compound (I)) (0.124 g, 48% yield). ¹H NMR (CD₃OD) δ 8.72 (m, 1H), 8.34 (s, 1H), 8.10 (dd, 1H), 7.67 (dd, 1H), 7.62 (dt, 1H), 7.58 (m, 1H), 7.47 (t, 1H), 7.04 (m, 1H), 4.27 (t, 2H), 2.89 (t, 2H), 2.63 (m, 4H), 1.68 (m, 4H), 1.51 (m, 2H). ES-MS (m/z) 389 [M+1]⁺.

### 6.2 Example 2

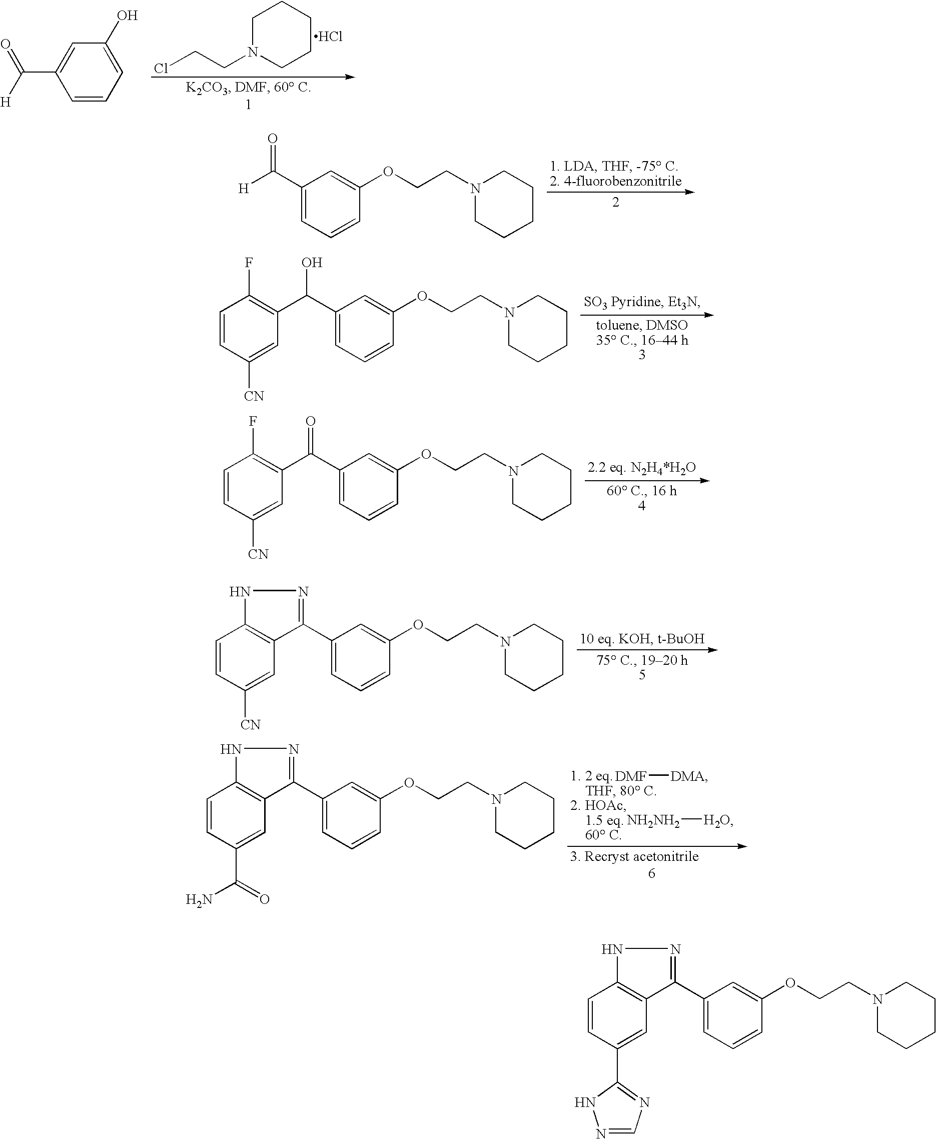

Step 1

A 600 L reactor was cleaned according to cGMP and purged with N₂. The reactor was charged with DMF under an inert N₂ atmosphere and 3-hydroxybenzaldehyde and K₂CO₃ were added. Chloroethyl piperidine-HCl was added at about 20 to about 28° C. over about 23 min and the reaction mixture was warmed to about 50° C. and stirred for about 15 hours at this temperature.

The reaction mixture was cooled to about 21° C. over about 75 min and the mixture was filtered (filtration time: about 45 min/about 2 bar). The filter cake was washed with TBME three times. The combined filtrates were quenched with half-sat. sodium carbonate solution causing the temperature to rise from about 20 to about 30° C. Water was added and the layers were separated. Due to the precipitation of salts, additional water was added to the aqueous layer. The original filter cake was washed with TBME three times. Each of the washings was used to extract the aqueous phase. Finally, the aqueous phase was extracted two times with TBME. The combined organic layers were washed with half-sat. sodium carbonate solution, NaOH solution, and water. The organic layer was filtered over Na₂SO₄ and the filter cake was washed with TBME. The organic solvent was removed at a jacket temperature of about 50° C. and reduced pressure (about 300 to about 70 mbar). The temperature increased from about 25 to about 49° C. during the distillation. An orange oil was obtained (57.936 kg; 93% yield; 100% purity by HPLC; 2.11% w/w of residual solvents by ¹H-NMR).

Step 2

A 100 L cryostat and a 160 L reactor were cleaned according to cGMP and purged with N₂. LDA was added to THF in a 100 L cryostat (inner temperature of about −80° C.), followed by the addition of 4-fluorobenzonitrile dissolved in THF at about −75 to about −80° C. during about 32 min (exothermic). After about 33 min, 3-(2-(piperidin-1-yl)ethoxy)benzaldehyde dissolved in THF was added over about 74 min at about −77 to about −79° C. (exothermic). Stirring was continued at about −80° C. for about 1 h. Conversion was estimated to be about 84% by HPLC.

The cold reaction mixture was transferred into a 160 L reactor charged with water (inner temperature of about 5° C.). The mixture was warmed to ambient temperature (about 18-about 22° C.) and extracted with TBME twice. 1 N HCl solution was added to the combined organic layers and the pH was adjusted to about 1-2 by addition of conc. HCl. The phases were separated and the organic layer was extracted with 1 N HCl solution twice. The combined acidic aqueous layers were washed with TBME twice. After adjusting the pH to about 10 by addition of 30% NaOH solution, the crude product was re-extracted with TBME twice. Additional 30% NaOH solution was added during the second extraction to readjust the pH to about 9.5. The combined TBME layers were washed with half-sat. NH₄Cl solution four times, 1 N NaOH solution twice and sat. NaCl solution. Solvent was removed at about 50° C. jacket temperature and about 400-about 310 mbar (inner temperature increased from about 26 to about 33° C.). The first portion of toluene was added and another 75 L of solvent were removed at about 60° C. jacket temperature. This was repeated a second time. Due to the precipitation of product upon cooling additional toluene was added. A clear, orange solution of 3-((3-(2-(piperidin-1-yl)ethoxy)phenyl)(hydroxy)methyl)-4-fluorobenzonitrile in toluene was obtained (166.5 kg; 71% yield, 82.88% purity by HPLC; loss on drying of 56%).

Step 3

A 600 L reactor was cleaned and purged with N₂. The scrubber was loaded with bleach and water. The reactor was charged with the toluene solution of 3-((3-(2-(piperidin-1-yl)ethoxy)phenyl)(hydroxy)methyl)-4-fluorobenzonitrile. Triethylamine and DMSO were added. SO₃.Py was added over about one hour at about 35 to 37° C. The reaction mixture was stirred at about 35° C. overnight. Conversion was estimated to be <5 mol % by NMR.

The clear solution was cooled to about 22° C. and NaOH solution 1 N was added over about two hours to the reaction mixture causing the temperature to increase from about 22 to about 25° C. while cooling (jacket temperature decreased from about 10 to about −25° C.). Isopropyl acetate was added and the pH was adjusted to about 12 by addition of NaOH solution 30% (exothermic). Water was added to the aqueous phase to dissolve most of the precipitated salts and the layers were separated. The aqueous phase was extracted with isopropyl acetate twice. All organic layers were combined and washed with NaOH solution 0.1 N three times and with sat. NaCl solution. Solvent was removed in vacuo (about 130 to about 50 mbar) at about 60° C. jacket temp. (inner temperature increased from about 25 to about 35° C. ). A first portion of toluene was added and about 93 L of solvent were distilled. A second portion of toluene was added and about 97 L of solvent were distilled. Finally, another portion of toluene was added. A brownish-yellow, intensively smelling solution of the desired product in toluene was obtained (248.88 kg; 83% yield, 80.10% purity by HPLC; loss on drying of 74.8%).

Step 4

A 640 L reactor was cleaned and purged with N₂. The scrubber was loaded with bleach and water. The reactor was charged with a solution of the starting material in toluene. The mixture was distilled at about 60° C. jacket temperature under reduced pressure to remove residual isopropyl acetate. During the distillation a first portion of toluene was added, and a sample was taken. No residual isopropyl acetate was detected by NMR.

At about 50° C., hydrazine hydrate was added slowly to starting material in toluene. Exothermic reaction and accumulation was controlled by dosing the hydrazine hydrate over about 103 minutes (maximum inner temperature of about 55° C.). The mixture was heated to about 60° C. over about 60 min and stirred at this temperature overnight (about 13 hours).

After cooling the yellow suspension to about 0° C. over about 2 h and stirring at this temperature for about another 2 h, the precipitated product was filtered. The filter cake was washed with water three times and with toluene three times. The product was dried on the nutsch filter dryer in a stream of nitrogen for about 2.5 h and finally at about 60° C. jacket temperature and reduced pressure overnight (about 18 h) (37.369 kg; 75% yield; 99.41% purity by HPLC; 0.04% w/w H₂O content by Karl-Fischer titration).

Step 5

A 640 L reactor was cleaned and purged with N₂. The reactor was charged with starting material, KOH powder and tert-butanol. After stirring the suspension at about 80° C. for about 3 hours a second portion of KOH powder was added. Stirring was continued for about 2 h and an in process control sample was taken. Conversion was estimated to be 58% by HPLC.

After stirring the suspension at about 80° C. for about 1.5 hours, the third portion of KOH powder was added and stirring was continued overnight (about 14 h). Conversion was estimated to be 99% by HPLC.

The suspension was cooled to about 30° C. and THF was added. At a jacket temperature of about 60° C. and about 150 to about 75 mbar, 355 L of solvent were distilled. The mixture was cooled to about 25° C. Addition of water dissolved all solids. The layers were separated and the turbid aqueous phase was extracted with THF in two portions. About 120 L of solvent of the combined organic layers were distilled (about 150 to about 100 mbar, jacket temperature of about 60° C.).

A pH electrode was installed onto the reactor. The product layer was slowly added to water at about 48 to about 50° C. causing the product to crystallize. By adding 1 N HCl-solution the pH was kept between about 12.0 and about 12.3 during the addition. After complete addition the pH should be about 12. The suspension was cooled to about 2° C. over about 1 hour and stirred at this temperature for about 30 minutes. The product was filtered under N₂ pressure. The filter cake was washed with water three times and TBME three times. The product was dried on the nutsch filter dryer in a stream of nitrogen for about 1 h and finally at about 50° C. jacket temperature and reduced pressure overnight (about 15 h) (35.816 kg; 91% yield; 98.97% purity by HPLC; 0.11% w/w H₂O content by Karl-Fischer titration).

Step 6

A 640 L reactor was cleaned and purged with N₂. 3-(3-(2-(piperidin-1-yl)ethoxy)phenyl)-1H-indazole-5-carboxamide was suspended in THF and dimethylformamid-dimethylacetal was added at about 22° C. The reaction mixture was stirred at about 64° C. for about three hours resulting in a clear solution, which was cooled to about 16° C. overnight. Conversion was estimated to be >99% by HPLC.

At a jacket temperature of about 60° C. and reduced pressure, 350 L of solvent were distilled. Dichloro-methane and water were added at an inner temperature of about 27° C. and the layers were separated. Approximately 166 L of the organic layer were distilled (about 60° C. jacket temperature, reduced pressure). TBME was added and the distillation was continued (about 115 L of distillate). A second portion of TBME was added. The resulting suspension was stirred at about 53 to about 55° C. for about I hour, cooled to about 1° C., and stirred for about 15 minutes at this temperature. The precipitated intermediate was collected by filtration and washed with TBME. The intermediate was dried on the nutsch filter dryer in a stream of nitrogen for about 1 h and finally at about 50° C. jacket temperature and reduced pressure overnight (about 12 h) (38.841 kg isolated).

Hydrazine monohydrate was added to acetic acid and THF at about 25 to about 30° C. over about 20 minutes. 38.841 kg of the intermediate were added over about 20 min at about 42 to about 49° C. The reaction mixture was stirred at about 67° C. inner temperature (reflux) for about 4.5 h. Conversion was estimated to be 97% by HPLC.

Stirring was continued for about 1.5 h at about 67° C. inner temperature. Conversion was estimated to again be 97% by HPLC.

Stirring was continued overnight (about 13 h) at about 67° C. inner temperature. Conversion was estimated to be 99.5% by HPLC.

At an internal temperature of about 25° C., 25% NH₃-solution was added (over about 2.5 h) to adjust the pH to about 10. The layers were separated and the aqueous phase was extracted twice with THF. Reactor und feed tanks were rinsed with water and afterwards with inline-filtered THF. The nutsch filter dryer (without filter cloth) was rinsed with inline-filtered acetonitrile. Afterwards a new filter cloth was installed. The clean reactor was charged with the combined, inline-filtered organic layers (355 L). At a jacket temperature of about 60° C. and reduced pressure, about 162 L of solvent were distilled. The product solution was stirred for about 5 days at about 20° C.

Inline-filtered acetonitrile was added to the solution and the resulting suspension was heated. At about 60° C. a clear solution was formed. The clear solution was cooled to about 52° C. inner temperature and seeded with a suspension of 1-(5-(1H-1,2,4-triazol-5-yl)(1H-indazol-3-yl))-3-(2-piperidylethoxy)benzene in inline-filtered acetonitrile. At a jacket temperature of about 60° C. and reduced pressure, the distillation was started. The distillation was carried out with an inner temperature controlled at ≧ about 40° C. Upon starting the distillation, a suspension formed immediately. About 360 L of solvent were distilled and inline-filtered acetonitrile was added in parallel to maintain a constant volume of the mixture. 1.6 mol % THF was estimated to be present by NMR.

Additional inline-filtered acetonitrile was added and the distillation was restarted. About 40 L of solvent were distilled. 1.2 mol % THF was estimated to be present by NMR.

Additional inline-filtered acetonitrile was added and about 40 L of solvent were removed by distillation. 0.8 mol % THF was estimated to be present by NMR.

The suspension was stirred at about 52° C. inner temperature overnight (about 10 h), cooled to about 20° C. over about one hour, and stirred at this temperature for about one hour. Crude product was collected by filtration and the filter cake was washed with inline-filtered acetonitrile in two portions. The product was dried on the nutsch filter dryer in a stream of nitrogen for about 1 h and finally at about 50° C. jacket temperature and reduced pressure for about 28 h (34.071 kg; 90% yield; 99.68% purity by HPLC).

### 6.3 Example 3

Isolation of Form A

A 2-L 3-necked round bottom flask equipped with a mechanical stirrer, vacuum-distillation set-up and thermometer was charged with amorphous Compound 1 (98.4 g), THF (490 mL, 5.0 vol.), and acetonitrile (490 mL, 5.0 vol). The stirred slurry was heated to about 65-70° C. and the heating mantle was immediately removed once the target temperature of about 65-70° C. was reached. The stirred solution was then cooled to about 50-53° C. and seeded with Form A (0.95 g in 10 mL acetonitrile). The stirred slurry was then vacuum distilled to remove about 500 mL of distillate. The distillation was carried out a pressure of about 320 torr to about 600 torr with the temperature being maintained between about 40° C. and 55° C. The stirred slurry was then charged with acetonitrile (490 mL, 5.0 vol) followed by vacuum distillation to remove about 500 mL of distillate. This distillation was carried out a pressure of about 320 torr to about 600 torr with the temperature being maintained between about 40° C. and 50° C. The stirred slurry was again charged with acetonitrile (735 mL, 7.5 vol.) and stirred at about 50-52° C. for about 16 hours. The stirred slurry was then allowed to cool to ambient temperature (about 22-25° C.) and allowed to stir at ambient temperature for about 30 minutes. The resulting solid was then collected using vacuum filtration, washed with acetonitrile (250 mL, 2.5 vol.) and dried at about 60° C. in vacuo for about 18 hours, to provide Form A as an off-white material in about 90% overall yield (89.7 g). HPLC, ¹H-NMR and ¹³C-NMR of the product were all identical to that of starting material.

| | | | | DVS analysis |
|---|---|---|---|---|
| | | | | (at 25° C., |
| | Melting | DSC | | below |
| Strong XRPD | Point | endotherm(s) | TGA | 75% relative |
| peaks | ° C. | ° C. | analysis | humidity) |
| 7.3, 15.2, 17.7, | 140 | 138 | Unsolvated | Not |
| 18.3, 21.2, 24.5 | | | | hygroscopic |

### 6.4 Example 4

Isolation of Form B

Compound (I) was isolated as Form B from recrystallization of Form A from acetone.

| | | | | DVS |
|---|---|---|---|---|
| | | | | analysis |
| | | | | (at 25° C., |
| Strong | Melting | DSC | | below |
| RPD | Point | endotherm(s) | TGA | 75% relative |
| peaks | ° C. | ° C. | analysis | humidity) |
| 6.5, 8.5, 8.9, | 137 | 137 | Unsolvated | Not |
| 14.9, 15.9, 18.0, | | | | hygroscopic |
| 19.0, 19.6, 24.9 | | | | |

### 6.5 Example 5

Isolation of Form C

Compound (I) was isolated as Form C from recrystallization of Form A from 2-propanol.

| | | | | DVS analysis |
|---|---|---|---|---|
| Strong | Melting | DSC | | (at 25° C., below |
| XRPD | Point | endotherm(s) | TGA | 75% relative |
| peaks | ° C. | ° C. | analysis | humidity) |
| 8.6, 11.8, 18.0, | 108 | 108 | 1:1 | Not |
| 21.9, 26.0 | | | Form C: | hygroscopic |
| | | | 2-propanol | |

### 6.6 Example 6

Isolation of Form D

Compound (I) was isolated as Form D from recrystallization of Form A from n-butyl acetate.

| | | | | DVS analysis |
|---|---|---|---|---|
| | | | | (at 25° C., |
| Strong | Melting | DSC | | below |
| XRPD | Point | endotherm(s) | TGA | 75% relative |
| peaks | ° C. | ° C. | analysis | humidity) |
| 5.0, 10.2, 12.2, | 138 | 138 | Unsolvated | Not |
| 15.2, 16.2, 18.0, | | | | hygroscopic |
| 19.6, 20.9, 23.7 | | | | |

### 6.7 Example 7

Isolation of Form E

Compound (I) was isolated as Form E from recrystallization of Form A from toluene.

| | | | | DVS analysis |
|---|---|---|---|---|
| | | | | (at 25° C., |
| Strong | Melting | DSC | | below |
| XRPD | Point | endotherm(s) | TGA | 75% relative |
| peaks | ° C. | ° C. | analysis | humidity) |
| 7.4, 15.3, 18.3, | 137 | 137 | Unsolvated | Slightly |
| 21.2, 24.5 | | | | hygroscopic |

### 6.8 Example 8

Isolation of Form F

Compound (I) was isolated as Form F from recrystallization of Form A from methyl t-butyl ether.

| | | | | DVS |
|---|---|---|---|---|
| | | | | analysis |
| | | | | (at 25° C., |
| Strong | Melting | DSC | | below |
| XRPD | Point | endotherm(s) | TGA | 75% relative |
| peaks | ° C. | ° C. | analysis | humidity) |
| 5.0, 9.9, 16.1, | 126 | Broad double | 2.5:1 | Not |
| 19.7, 25.8 | | beginning at | Form F: | hygroscopic |
| | | 126 | Methyl t-butyl | |
| | | | ether | |

### 6.9 Example 9

Isolation of Form G

Compound (I) was isolated as Form G from recrystallization of Form A from methyl ethyl ketone.

| | | | | DVS analysis |
|---|---|---|---|---|
| | | | | (at 25° C., |
| Strong | Melting | DSC | | below |
| XRPD | Point | endotherm(s) | TGA | 75% relative |
| peaks | ° C. | ° C. | analysis | humidity) |
| 4.9, 9.7, 16.4, | 134 | 134 | 7:1 | Slightly |
| 19.8, 20.0, 26.2 | | | Form G: | hygroscopic |
| | | | Methyl ethyl | |
| | | | ketone | |

### 6.10 Example 10

Isolation of Form H

Compound (I) was isolated as Form H from recrystallization of Form A from tetrahydrofuran/water.

| | | | | DVS analysis |
|---|---|---|---|---|
| | | | | (at 25° C., |
| Strong | Melting | DSC | | below |
| XRPD | Point | endotherm(s) | TGA | 75% relative |
| peaks | ° C. | ° C. | analysis | humidity) |
| 4.8, 9.7, 16.2, | 119 | 119 | 5:1 | Hygroscopic |
| 19.6, 26.0 | | | Form H: | |
| | | | tetrahydrofuran | |

### 6.11 Example 11

Isolation of Form I

Compound (I) was isolated as Form I from recrystallization of Form A from ethanol.

| | | | | DVS analysis |
|---|---|---|---|---|
| Strong | Melting | DSC | | (at 25° C., below |
| XRPD | Point | endotherm(s) | TGA | 75% relative |
| peaks | ° C. | ° C. | analysis | humidity) |
| 8.8, 17.6, 18.8, | 98 | 98 | 1:1 | Not |
| 19.2, 21.2, 24.3, | | | Form I: | hygroscopic |
| 26.4, 29.0 | | | ethanol | |

### 6.12 Example 12

Isolation of Form J

Compound (I) was isolated as Form J from recrystallization of Form A from methyl ethyl ketone/heptane or methyl ethyl ketone/toluene.

| | | | | DVS analysis |
|---|---|---|---|---|
| Strong | Melting | DSC | | (at 25° C., below |
| XRPD | Point | endotherm(s) | TGA | 75% relative |
| peaks | ° C. | ° C. | analysis | humidity) |
| 4.8, 12.0, 16.2, | 134 | 134 | 2.5:1 | Slightly |
| 17.6, 19.6, 20.0, | | | Form J: | hygroscopic |
| 23.7, 26.0 | | | heptane | |

### 6.13 Example 13

This example illustrates assays that can be used for evaluating the solid forms of the invention.

JNK Assay

To 10 μL of a solid form of the invention in 20% DMSO/80% dilution buffer consisting of 20 mM HEPES (pH 7.6), 0.1 mM EDTA, 2.5 mM magnesium chloride, 0.004% Triton x100, 2 μg/mL leupeptin, 20 mM β-glycerolphosphate, 0.1 mM sodium vanadate, and 2 mM DTT in water is added 30 μL of 50-200 ng His6-JNK1, JNK2 or JNK3 in the same dilution buffer. The mixture is preincubated for 30 minutes at room temperature. Sixty microliter of 10 μg GST-c-Jun(1-79) in assay buffer consisting of 20 mM HEPES (pH 7.6), 50 mM sodium chloride, 0.1 mM EDTA, 24 mM magnesium chloride, 1 mM DTT, 25 mM PNPP, 0.05% Triton x100, 11 μM ATP, and 0.5 μCi γ-32P ATP in water is added and the reaction is allowed to proceed for 1 hour at room temperature. The c-Jun phosphorylation is terminated by addition of 150 μL of 12.5% trichloroacetic acid. After 30 minutes, the precipitate is harvested onto a filter plate, diluted with 50 μL of the scintillation fluid and quantified by a counter. The IC₅₀ values are calculated as the concentration of the test solid form at which the c-Jun phosphorylation is reduced to 50% of the control value. Preferred solid forms of the present invention have an IC₅₀ value ranging 0.01-10 μM in this assay.

Jurkat T-cell IL-2 Production Assay

Jurkat T cells (clone E6-1) are purchased from the American Tissue Culture Collection and maintained in growth media consisting of RPMI 1640 medium containing 2 mM L-glutamine (Mediatech), with 10% fetal bovine serum (Hyclone) and penicillin/streptomycin. All cells are cultured at 37° C. in 95% air and 5% CO₂. Cells are plated at a density of 0.2×10⁶ cells per well in 200 μL of media. A stock solution of the solid form of the invention (20 mM) is diluted in growth media and added to each well as a 10× concentrated solution in a volume of 25 ÿL, mixed, and allowed to pre-incubate with cells for 30 minutes. The vehicle (dimethylsulfoxide) is maintained at a final concentration of 0.5% in all samples. After 30 minutes the cells are activated with PMA (phorbol myristate acetate; final concentration 50 ng/mL) and PHA (phytohemagglutinin; final concentration 2 μg/mL). PMA and PHA are added as a 10× concentrated solution made up in growth media and added in a volume of 25 μL per well. Cell plates are cultured for 10 hours. Cells are pelleted by centrifugation and the media removed and stored at −20° C. Media aliquots are analyzed by sandwich ELISA for the presence of IL-2 as per the manufacturers instructions (Endogen). The IC₅₀ values are calculated as the concentration of the test solid form at which the IL-2 production was reduced to 50% of the control value. Preferred solid forms of the present invention have an IC₅₀ value ranging 0.1-30 μM in this assay.

Mouse in vivo LPS-Induced TNF-α Production Assay

Non-fasted mice are acclimatized for at least 7 days. Groups of 4 to 6 female BALB/c or CD-1 mice (8-10 weeks of age from Charles River laboratories) are pretreated with a test solid form, either by intravenous injection or by oral gavage 15-180 minutes prior to the injection of 0.5 mg/kg Bacto LPS from E. coli 055:B5 (Difco Labs). Ninety minutes after LPS challenge, a terminal bleed is performed via abdominal vena cava and blood is allowed to clot at room temperature for 30 minutes in Microtainer serum separator tubes. After separation by centrifugation, the serum is stored frozen at −80° C. ELISA is performed on thawed, diluted samples (1:10 to 1:20) using a Mouse TNF-alpha kit (Biosource International). The ED₅₀ values are calculated as the dose of the test solid form at which the TNF-α production is reduced to 50% of the control value. Preferred solid forms of the present invention have an ED₅₀ value ranging 1-30 mg/kg in this assay.

Inhibition of Leukocyte Recruitment in Rat Inflamed Lung

Aerosol administration of ovalbumun in Brown Norway Rats previously sensitized by injection of ovalbumin (OA) results in an allergic airway inflammation marked by the generation of an eosinophil- and T-lymphocyte-rich leukocytic infiltration in the lungs (see Richards et al., Am. J. Physiol, 271:2 Pt 1, L267-76, 1996). A solid form of the invention is administered by subcutaneous injection at a dose of 30 mg/kg, b.i.d. for 3 days prior to ovalbumin challenge by aerosol. Cell counts are obtained from samples of bronchoalveolar lavage.

Rat In Vivo Adjuvant Arthritis

Male Lewis rats are immunized with complete Freund's adjuvant on day 0 to induce an aggressive arthritis characterized by joint destruction and paw swelling. A solid form of the invention is administered subcutaneously once daily from day 8 to day 20. Paw swelling is determined be water displacement plethysmometry. Radiographs are obtained of the right hind paw to assess bone changes using a semi-quantitative scoring system: demineralization (0-2+), calcaneal erosion (0-1+), and heterotropic bone formation (0-1+), with a maximum possible score=6 (see FIG. 4B). Activation of AP-1 is determined by DNA binding activity in an electrophoretic mobility shift assay (EMSA) (Ausubel et al., Short Protocols in Molecular Biology, Second Edition, John Wiley & Sons Publisher, New York, 1992). Matrix metalloproteinase-13 expression is measured by nothern blot analysis of MMP-13 mRNA (Ausebel et al., supra) (see also Winter et al., Arthritis and Rheumatism 9(3):394-404, 1966; Weichman et al., Pharmacological Methods in the Control of Inflammation, Chang and Lewis Eds., Alan R. Liss, Inc., Publ., New York, 1989).

Kainic Acid-Induced Seizure Response

A solid form of the invention is administered to male CD rats at 10 mg/kg intravenously through a tail vein catheter. This is followed immediately by a 30 mg/kg subcutaneous injection. Vehicle controls receive the same injection volumes of the PPCES vehicle alone. Thirty minutes later, animals are given a 1-mg/kg i.p. injection of kainic acid in normal saline solution. This dose of kainic acid has been previously reported to induce a seizure syndrome in rats (Maj et al., Eur. J. Pharm. 359:27-32, 1992). Seizure behavior is monitored for 4 hours following kainic acid injection.

It will be appreciated that, although specific embodiments of the invention have been described herein for purposes of illustration, various modifications may be made without departing from the spirit and scope of the invention. Accordingly, the invention is not limited except as by the appended claims. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A Form A solid form of the compound of formula (I):

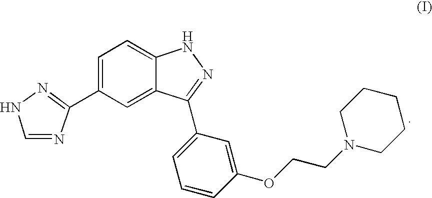

2. The solid form of claim 1 having x-ray powder diffraction peaks at 7.3, 15.2, 17.7, 18.3, 21.2 and 24.5.

3. The solid form of claim 1 having a differential scanning calorimetry melting temperature maximum of about 153° C.

4. The solid form of claim 1 that is obtained by crystallizing said compound of formula (I) from acetonitrile.

5. The solid form of claim 1 having a melting point of about 140° C.

6. A Form B solid form of the compound of formula (I):

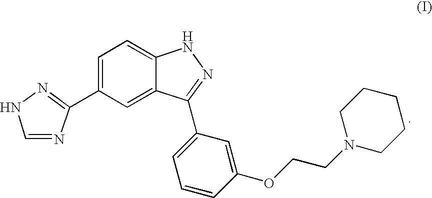

7. The solid form of claim 6 having x-ray powder diffraction peaks at 6.5, 8.5, 8.9, 14.9, 15.9, 18.0, 19.0, 19.6 and 24.9.

8. The solid form of claim 6 having a differential scanning calorimetry melting temperature maximum of about 149° C.

9. The solid form of claim 6 that is obtained by crystallizing said compound of formula (I) from acetonitrile.

10. The solid form of claim 6 having a melting point of about 137° C.

11. A Form C solid form of the compound of formula (I):

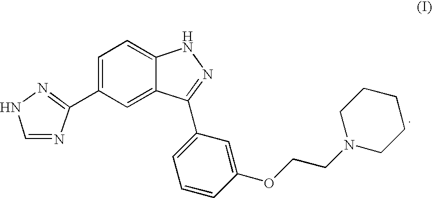

12. The solid form of claim 11 having x-ray powder diffraction peaks at 8.6, 11.8, 18.0, 21.9 and 26.0.

13. The solid form of claim 11 having a differential scanning calorimetry melting temperature maximum of about 125° C.

14. The solid form of claim 11 that is obtained by crystallizing said compound of formula (I) from acetonitrile.

15. The solid form of claim 11 having a melting point of 108° C.

16. A Form D solid form of the compound of formula (I):

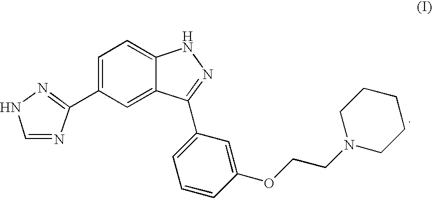

17. The solid form of claim 16 having x-ray powder diffraction peaks at 5.0, 10.2, 12.2, 15.2, 16.2, 18.0, 19.6, 20.9 and 23.7.

18. The solid form of claim 16 having a differential scanning calorimetry melting temperature maximum of about 150° C.

19. The solid form of claim 16 that is obtained by crystallizing said compound of formula (I) from acetonitrile.

20. The solid form of claim 16 having a melting point of 138° C.

21. A Form E solid form of the compound of formula (I):

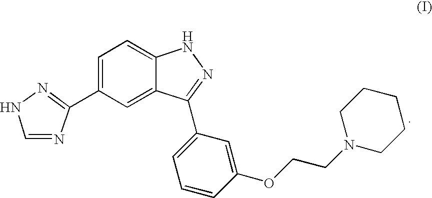

22. The solid form of claim 21 having x-ray powder diffraction peaks at 7.4, 15.3, 18.3, 21.2 and 24.5.

23. The solid form of claim 21 having a differential scanning calorimetry melting temperature maximum of about 152° C.

24. The solid form of claim 21 that is obtained by crystallizing said compound of formula (I) from acetonitrile.

25. The solid form of claim 21 having a melting point of 137° C.

26. A Form F solid form of the compound of formula (I):

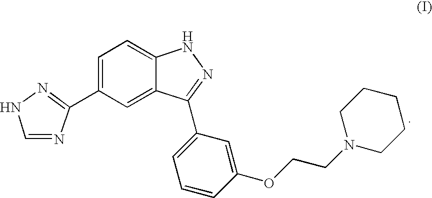

27. The solid form of claim 26 having x-ray powder diffraction peaks at 5.0, 9.9, 16.1, 19.7 and 25.8.

28. The solid form of claim 26 having a differential scanning calorimetry melting temperature maximum of about 142° C.

29. The solid form of claim 26 that is obtained by crystallizing said compound of formula (I) from acetonitrile.

30. The solid form of claim 26 having a melting point of 126° C.

31. A Form G solid form of the compound of formula (I):

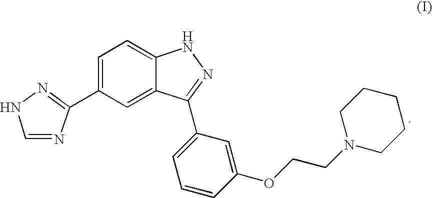

32. The solid form of claim 31 having x-ray powder diffraction peaks at 4.9, 9.7, 16.4, 19.8, 20.0 and 26.2.

33. The solid form of claim 31 having a differential scanning calorimetry melting temperature maximum of about 146° C.

34. The solid form of claim 31 that is obtained by crystallizing said compound of formula (I) from acetonitrile.

35. The solid form of claim 31 having a melting point of 134° C.

36. A Form H solid form of the compound of formula (I):

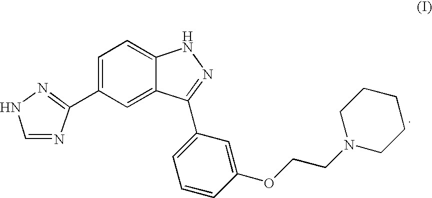

37. The solid form of claim 36 having x-ray powder diffraction peaks at 4.8, 9.7, 16.2, 19.6 and 26.0.

38. The solid form of claim 36 having a differential scanning calorimetry melting temperature maximum of about 129° C.

39. The solid form of claim 36 that is obtained by crystallizing said compound of formula (I) from acetonitrile.

40. The solid form of claim 36 having a melting point of 119° C.

41. A Form I solid form of the compound of formula (I):

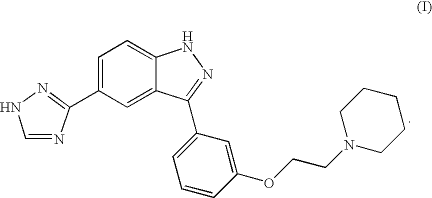

42. The solid form of claim 41 having x-ray powder diffraction peaks at 8.8, 17.6, 18.8, 19.2, 21.2, 24.3, 26.4 and 29.0.

43. The solid form of claim 41 having a differential scanning calorimetry melting temperature maximum of about 110° C.

44. The solid form of claim 41 that is obtained by crystallizing said compound of formula (I) from acetonitrile.

45. The solid form of claim 41 having a melting point of 98° C.

46. A Form J solid form of the compound of formula (I):

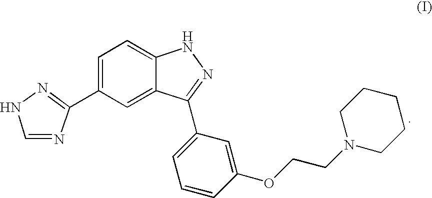

47. The solid form of claim 46 having x-ray powder diffraction peaks at 4.8, 12.0, 16.2, 17.6, 19.6, 20.0, 23.7 and 26.0.

48. The solid form of claim 46 having a differential scanning calorimetry melting temperature maximum of about 148° C.

49. The solid form of claim 46 that is obtained by crystallizing said compound of formula (I) from acetonitrile.

50. The solid form of claim 46 having a melting point of 134° C.

51. The solid form of claim 1, wherein the solid form is in a pure form.

52. A pharmaceutical composition comprising the solid form of claim 1 and a pharmaceutically acceptable carrier.

53. The pharmaceutical composition of claim 52 wherein the solid form is in a pure form.

54. A method for treating or preventing cancer in a patient in need thereof, comprising administering to the patient an effective amount of a solid form of claim 1.

55. The method of claim 54 wherein the cancer is of the head, neck, eye, mouth, throat, esophagus, chest, bone, lung, colon, rectum, stomach, prostate, breast, ovaries, testicles or other reproductive organs, skin, thyroid, blood, lymph nodes, kidney, liver, pancreas, brain or central nervous system.
